(19) United States
(12) Patent Application Publication
(10) Pub. No.: US 2006/0281083 A1
(43) Pub. Date: Dec. 14, 2006

(54) Identification of genes involved in angiogenesis, and development of an angiogenesis diagnostic chip to identify patients with impaired angiogenesis

(75) Inventors: Stephen E. Epstein, Rockville, MD (US); Mary Susan Burnett, Fairfax, VA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Medstar Research Institute, Washington D.C. (US)

(21) Appl. No.: 10/538,635

(22) PCT Filed: Dec. 10, 2003
(86) PCT No.: PCT/US03/38950

§ 371 Date: Jul. 17, 2006

Related U.S. Application Data
(60) Provisional application No. 60/432,005, filed on Dec. 10, 2002.

(51) Int. Cl. C12Q 1/68; C12P 19/34; A61K 48/00

(52) U.S. Cl. 435/6; 435/91.2; 514/44

(57) ABSTRACT

The invention is directed to methods for angiotyping individual patients to predict the likelihood of whether a given individual will develop good vs. poor collaterals naturally. Accordingly, this can involve obtaining and providing a list of genes involved in collateral development. In particular, angiotyping individual patients can be used to predict the likelihood of whether a given individual will develop good vs. poor collaterals in response to specific angiogenesis therapy. From an array of genes that have been determined through experimental studies as being differentially expressed in tissues in which collaterals are developing in response to arterial occlusion, single nucleotide polymorphisms (SNPs), or other epigenetic changes, such as DNA methylation patterns, can be identified. SNPs and DNA methylation patterns are detected using microchips or similar technology assaying for all, or most, of the genes determined to play a role in collateral development. In addition, abnormally low or abnormally high differential expression of any combination of the candidate genes can be detected in such tissue as peripheral blood cells. The presence of a predisposition to develop poor vs. good collaterals is indicated by the presence of SNPs, and/or alterations in DNA methylation patterns, and/or difference in expression levels involving one or more of the genes.

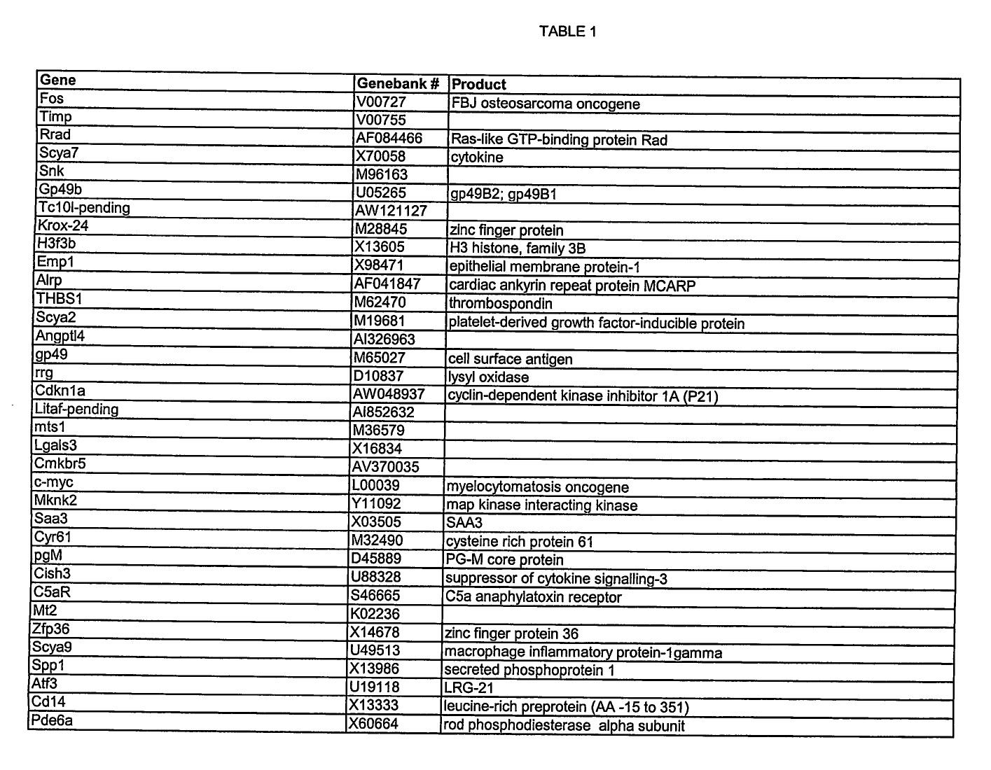

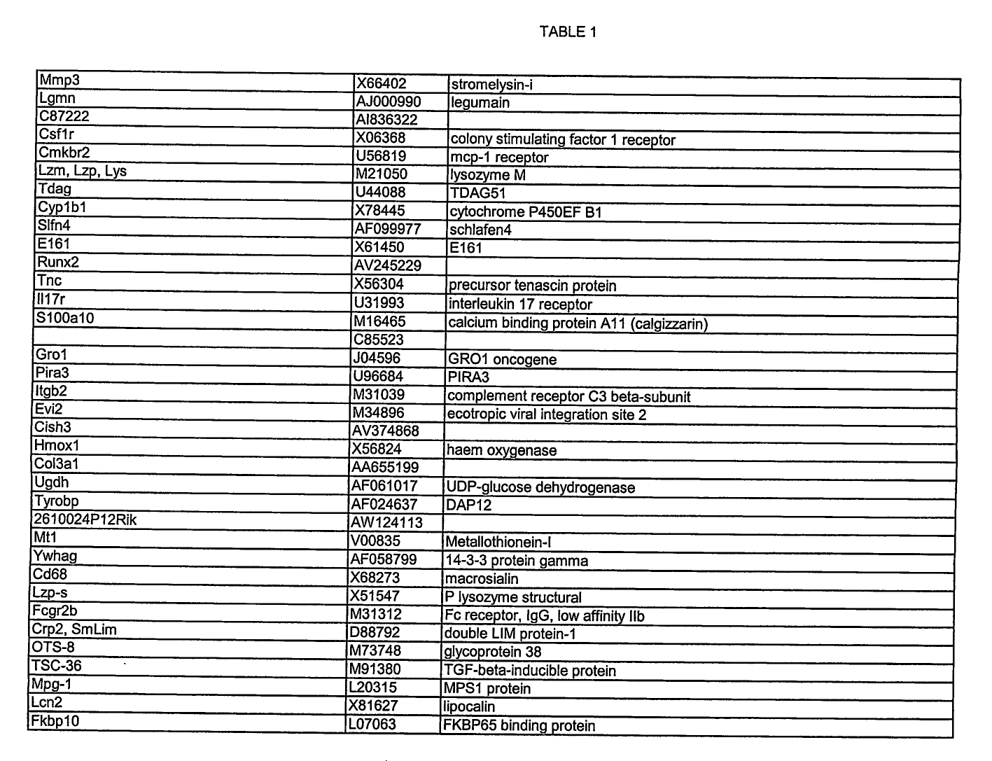

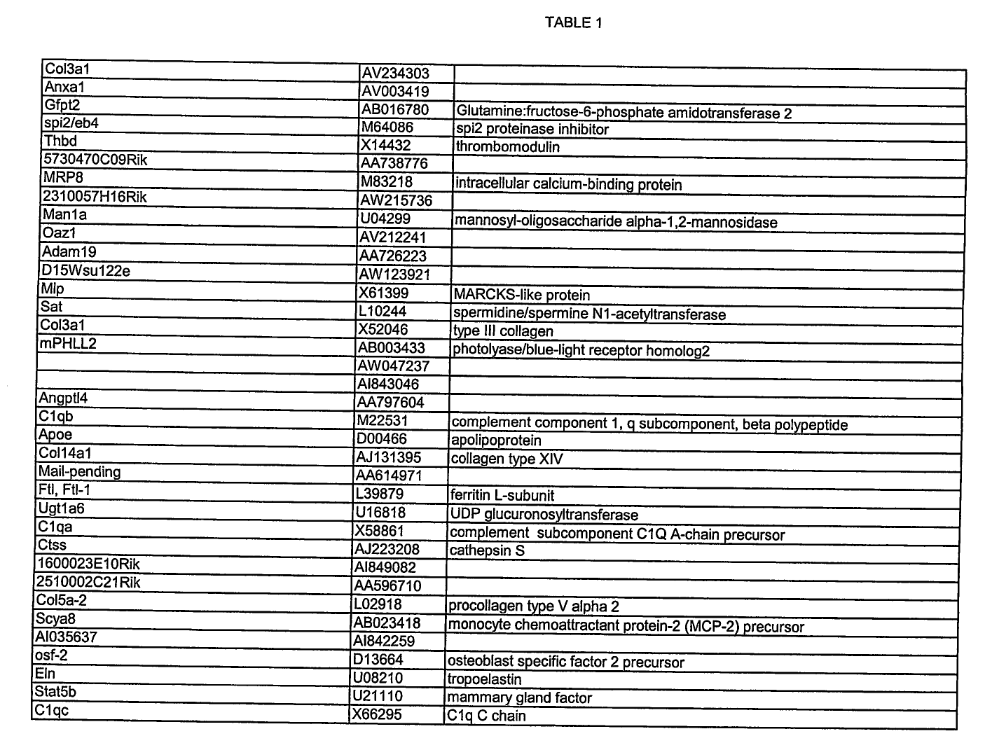

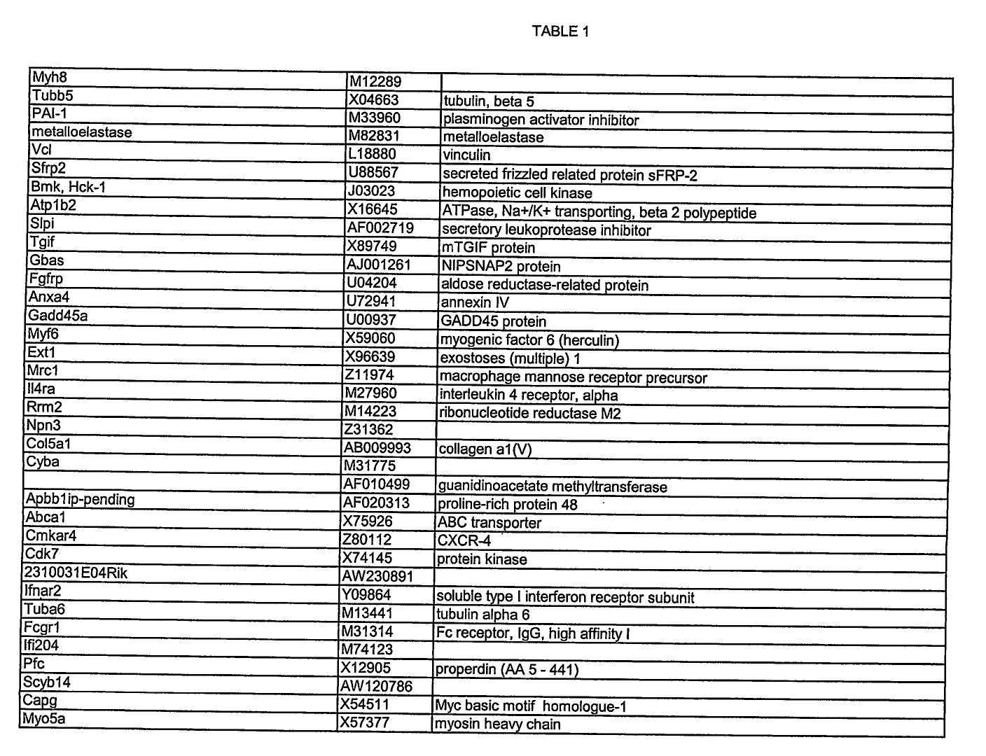

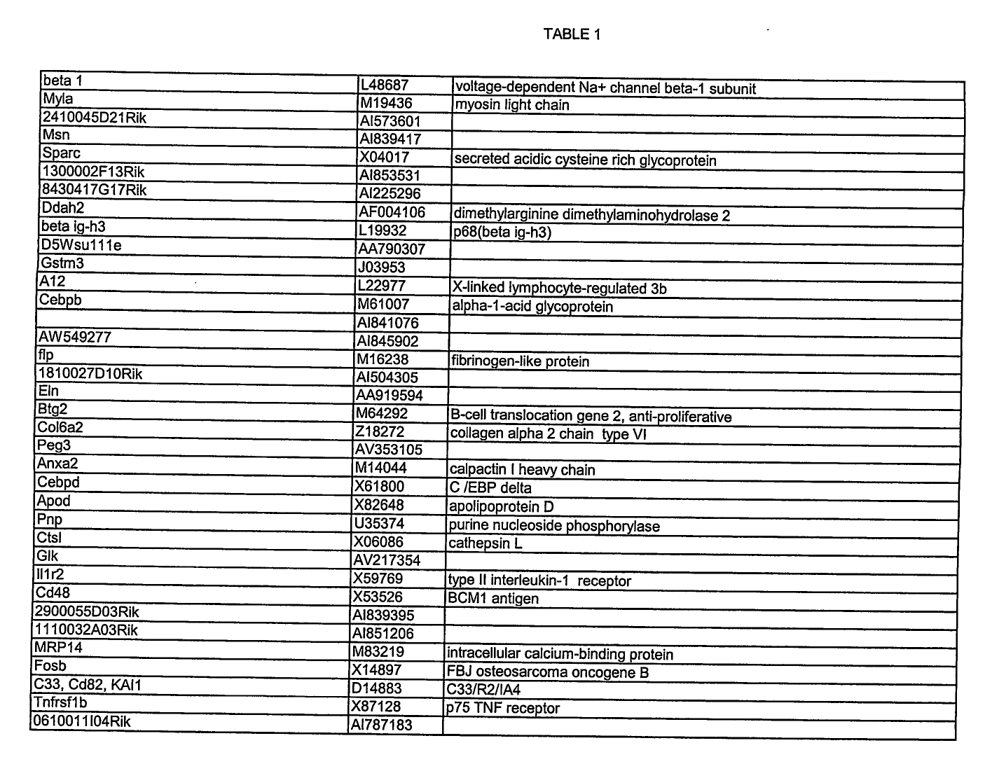

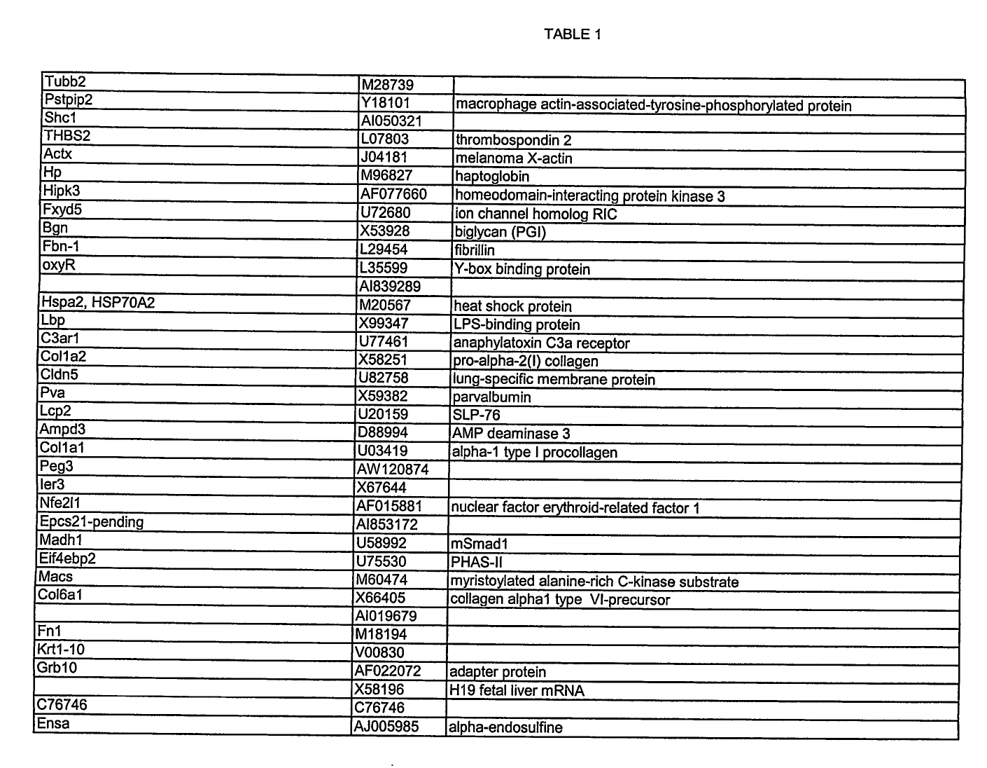

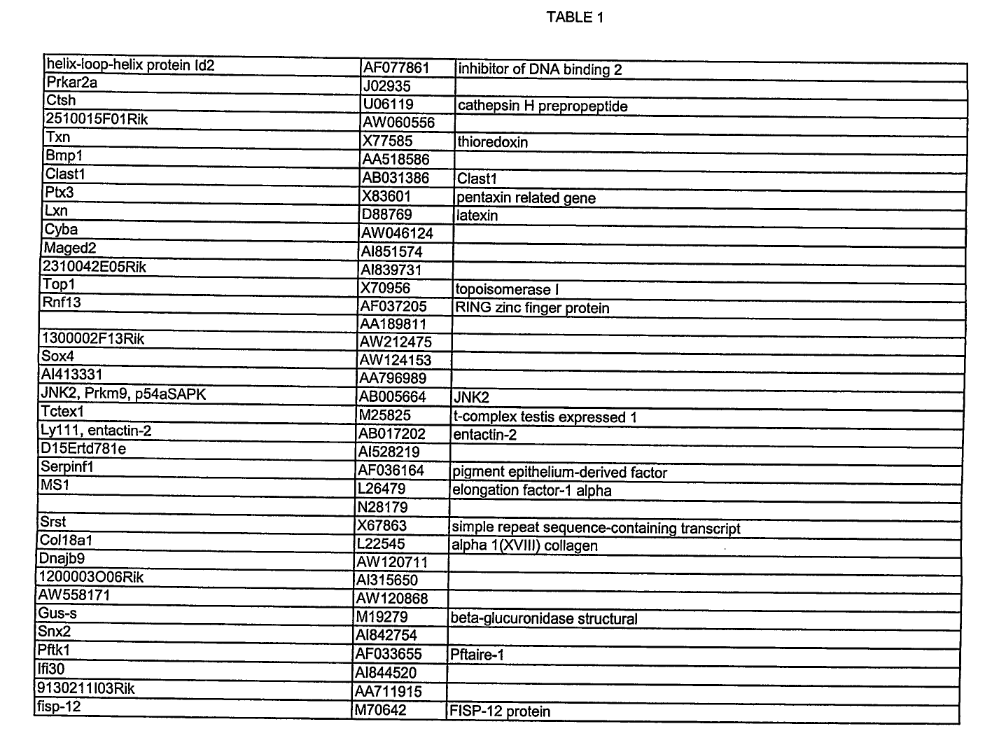

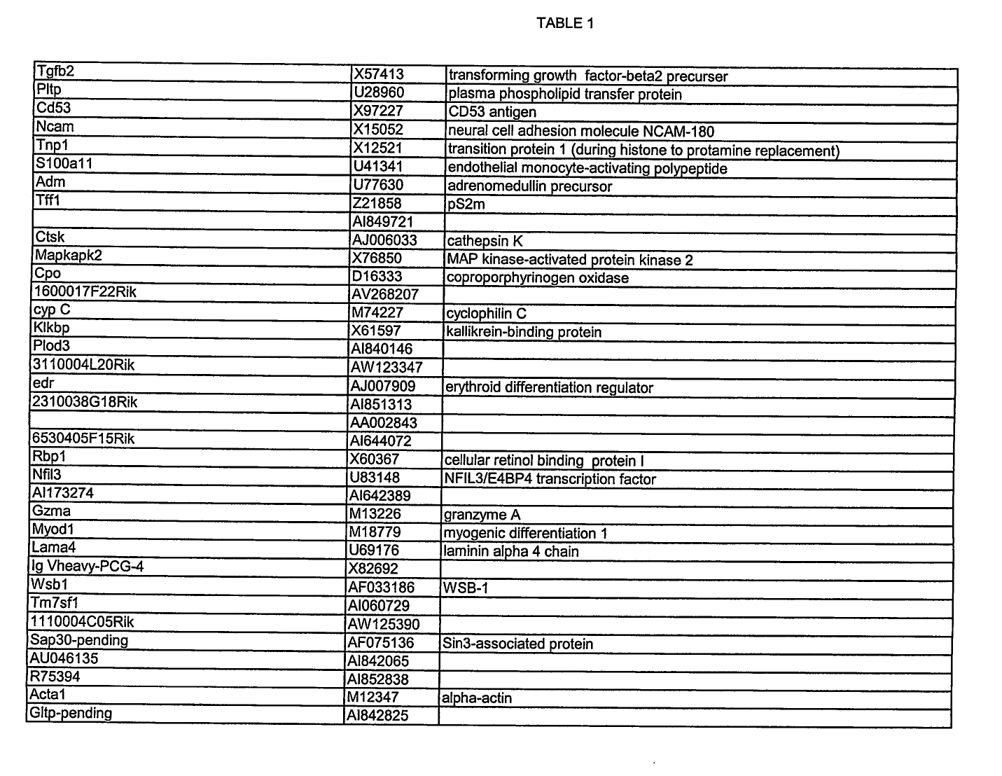

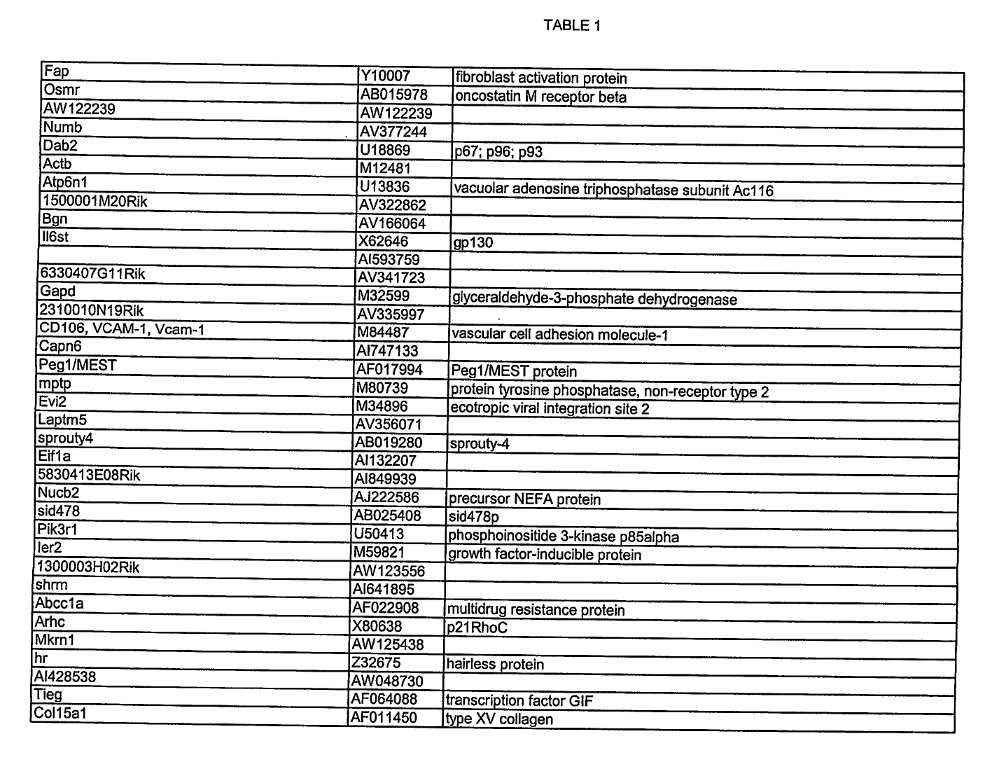

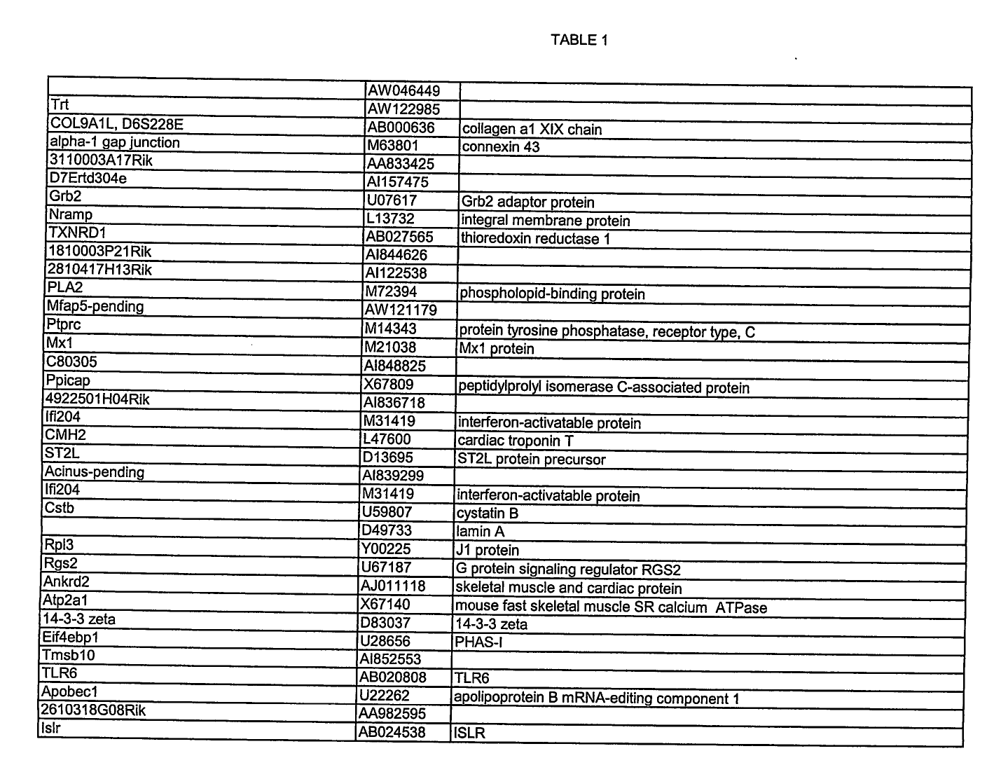

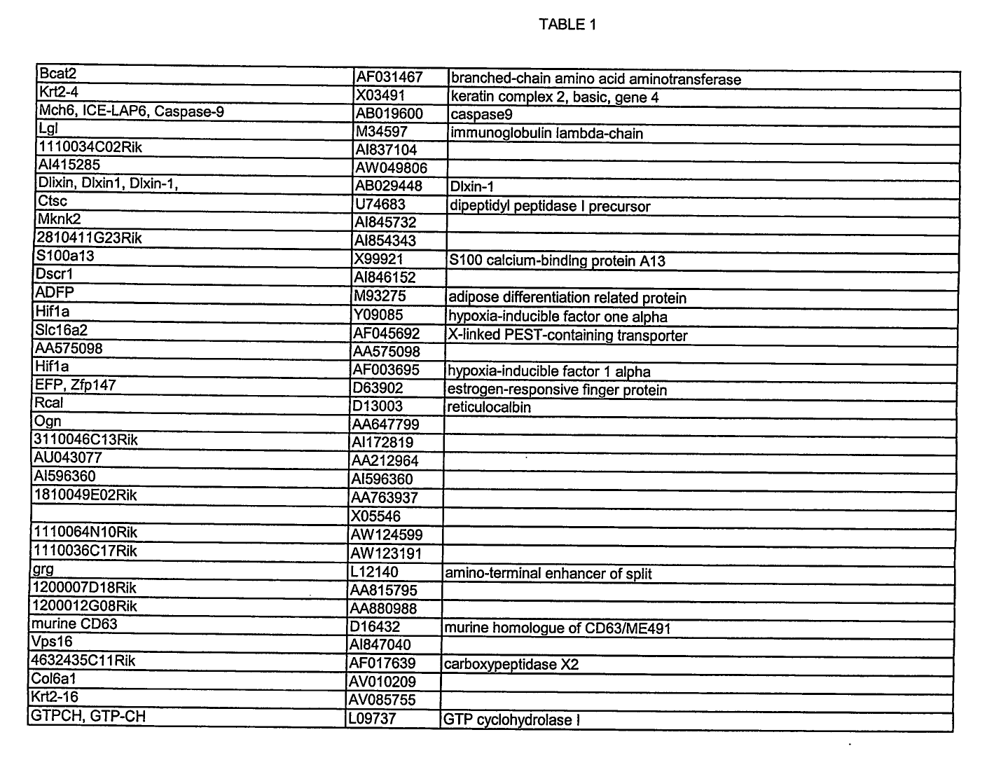

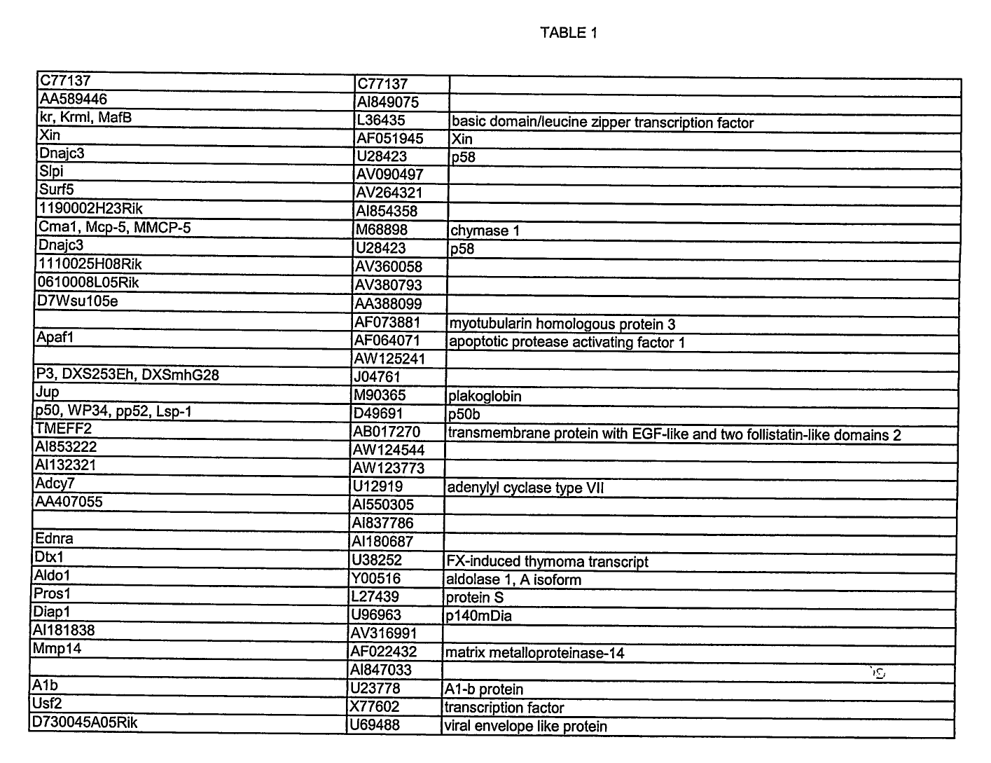

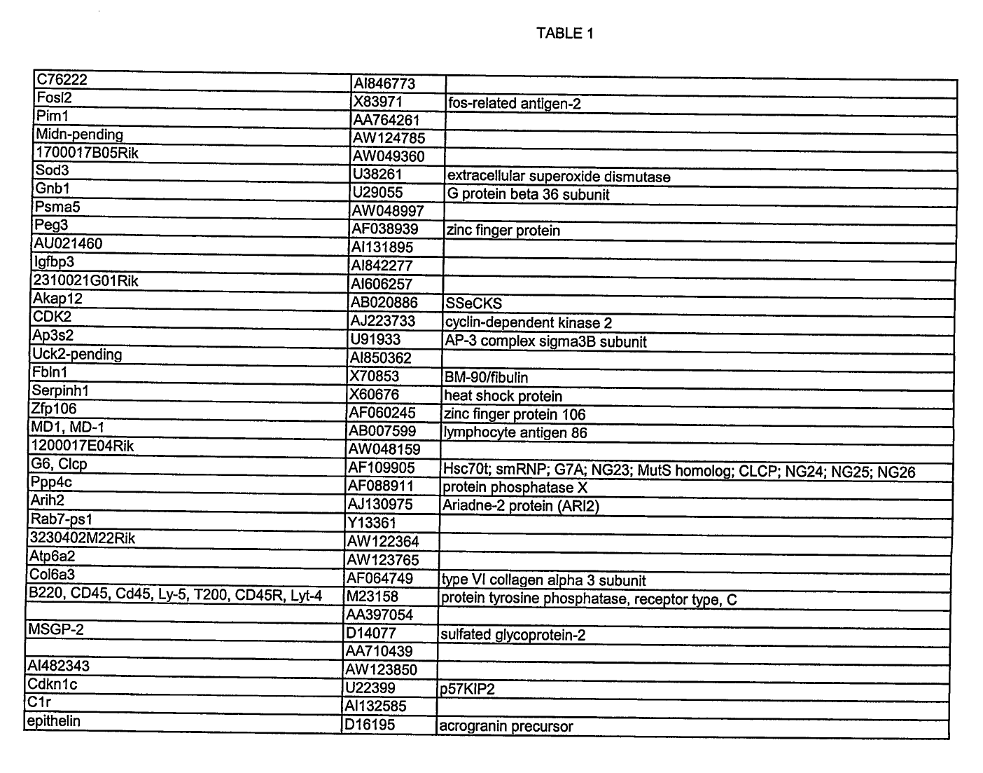

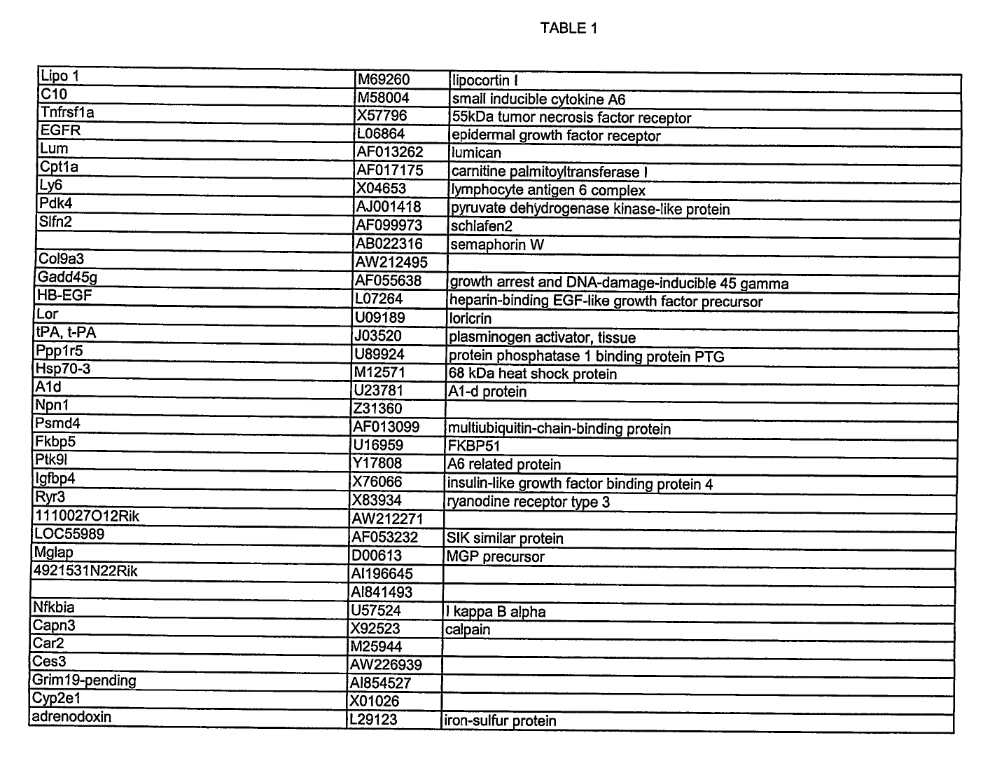

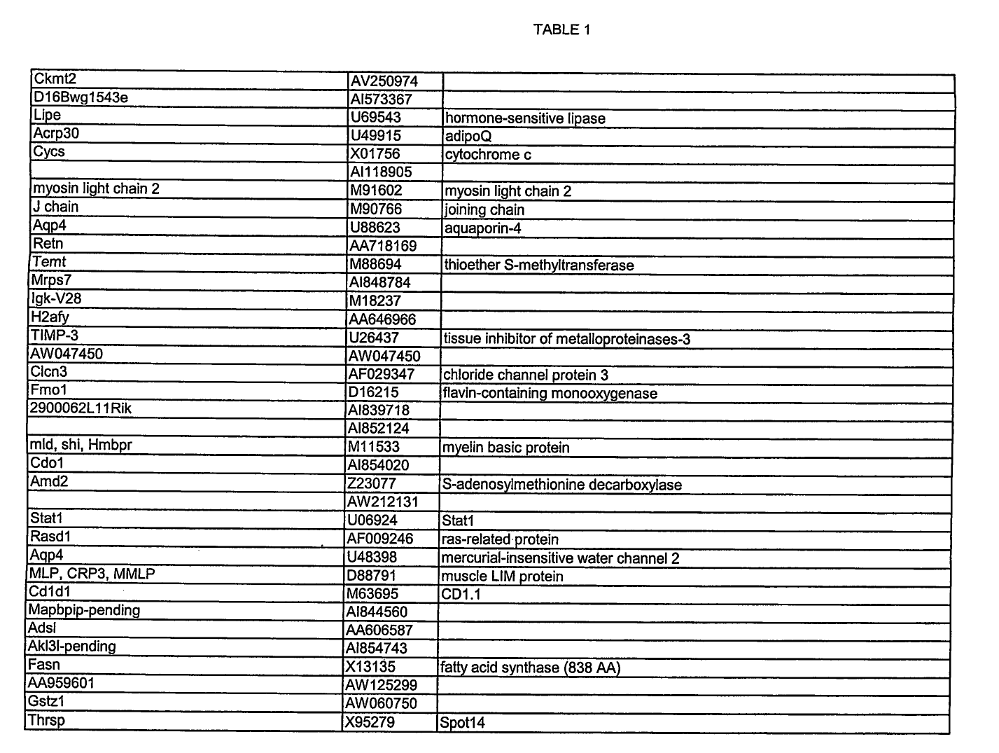

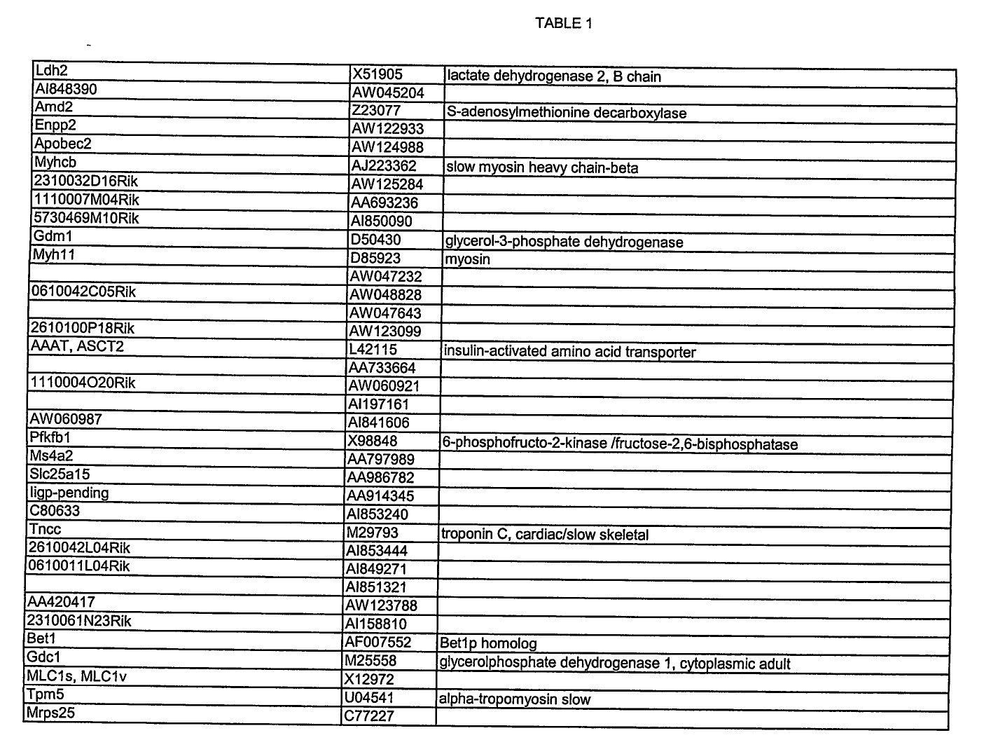

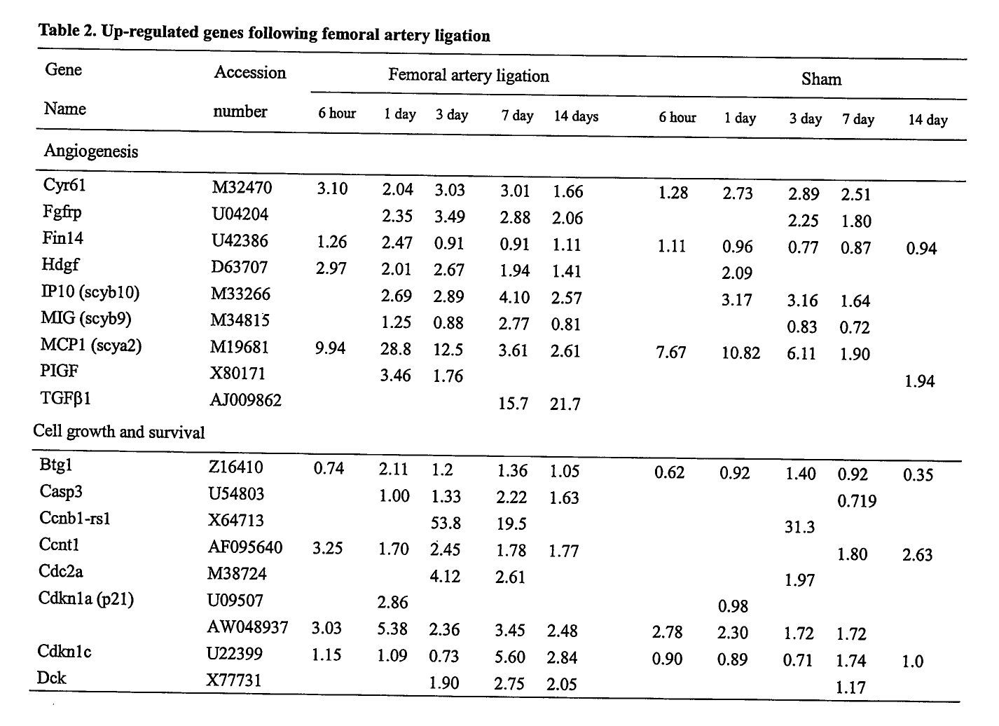

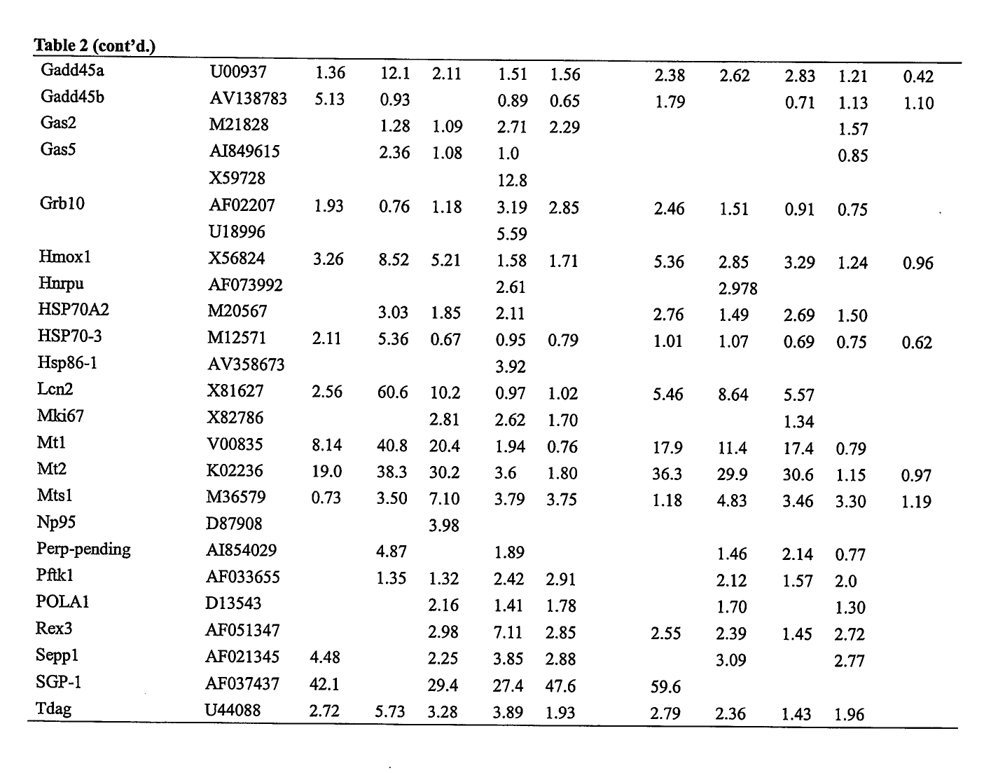

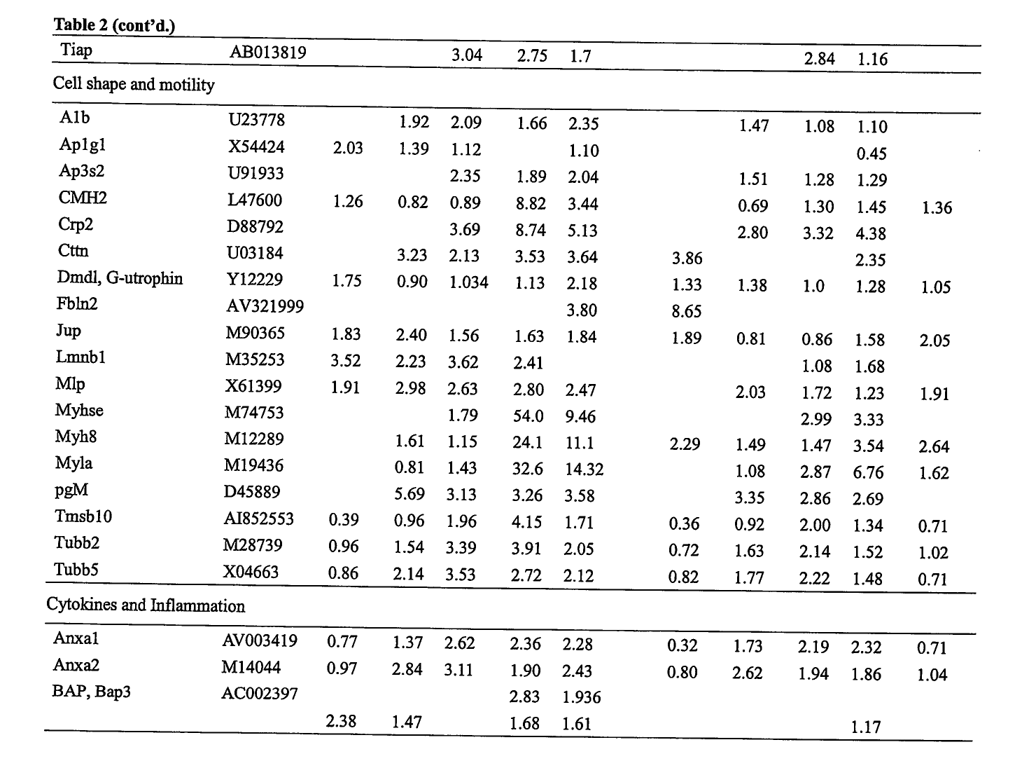

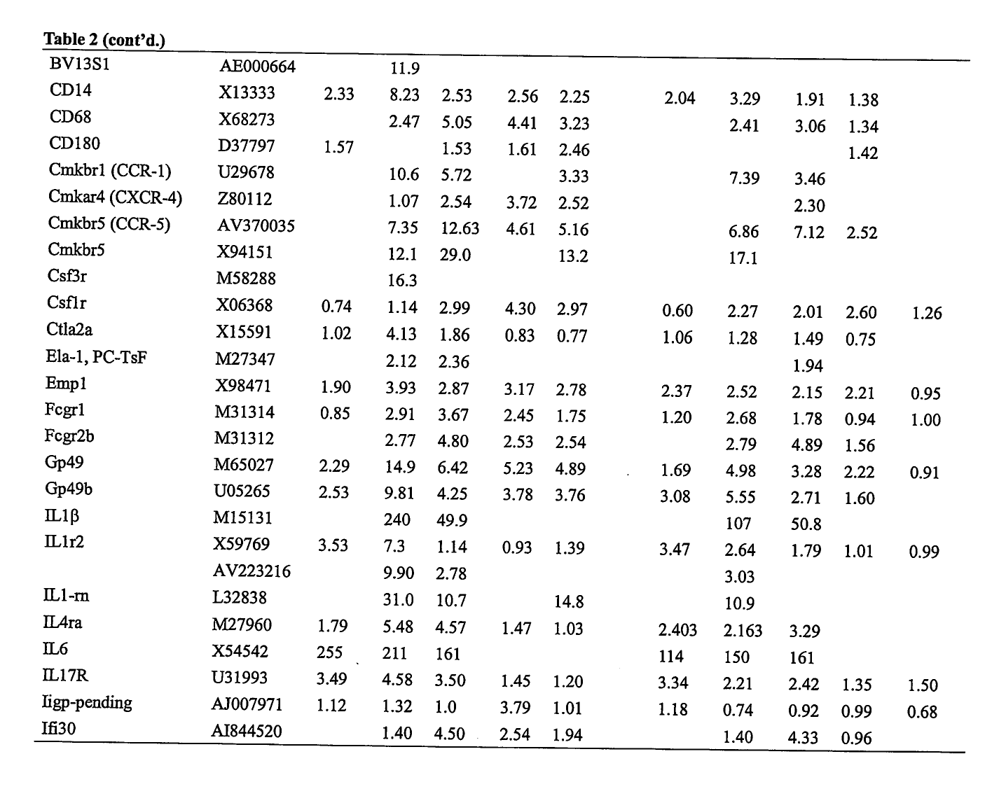

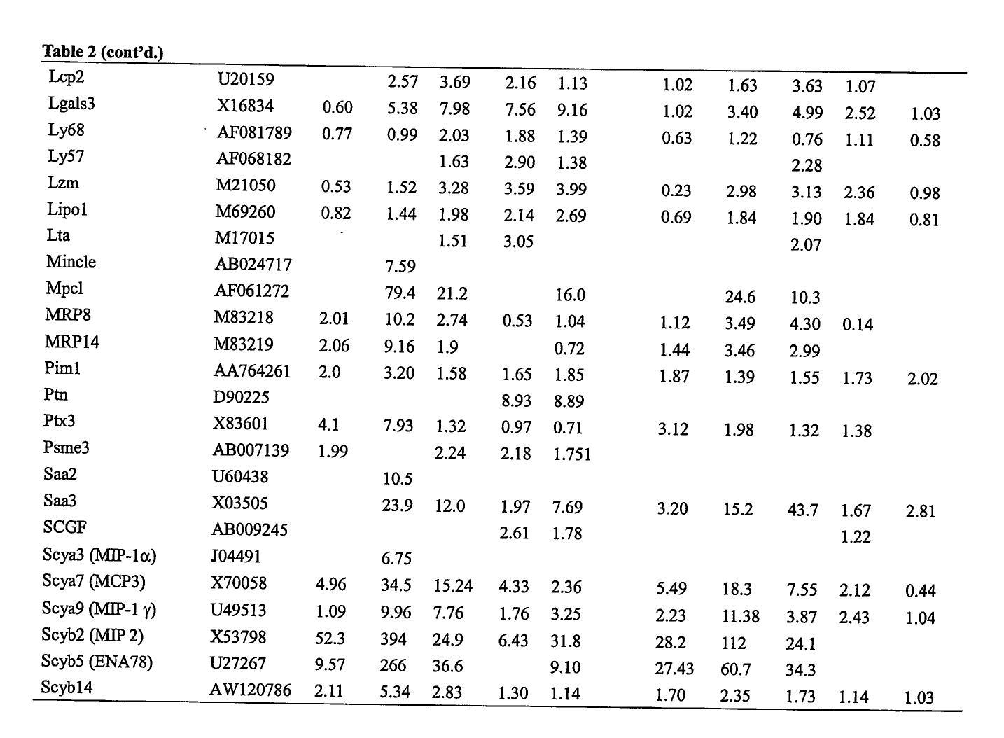

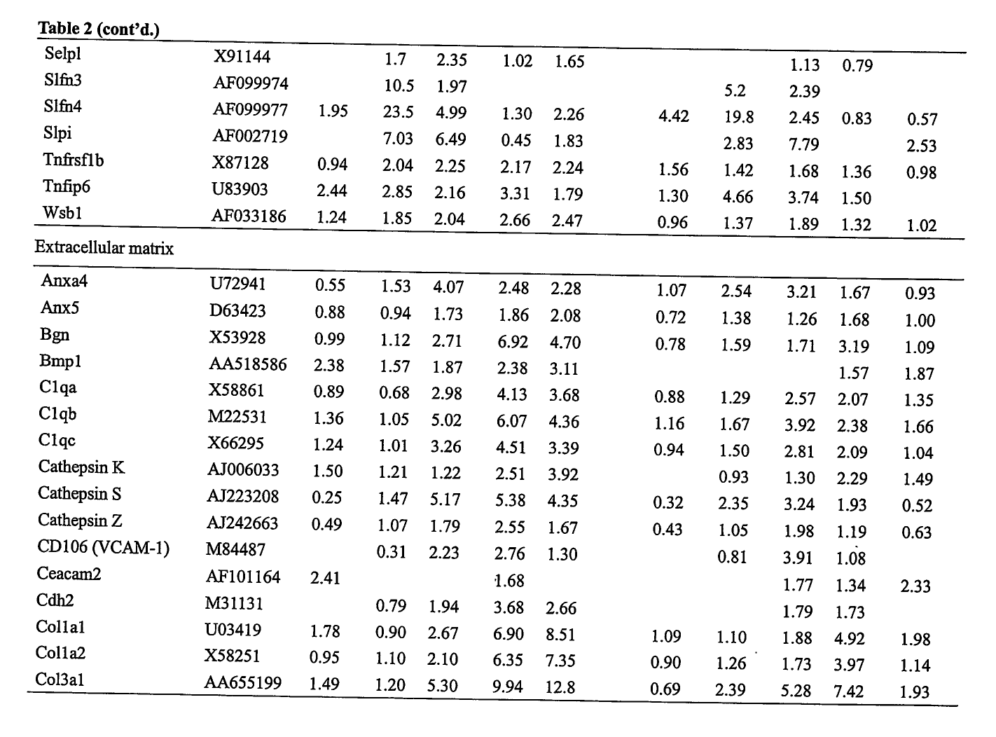

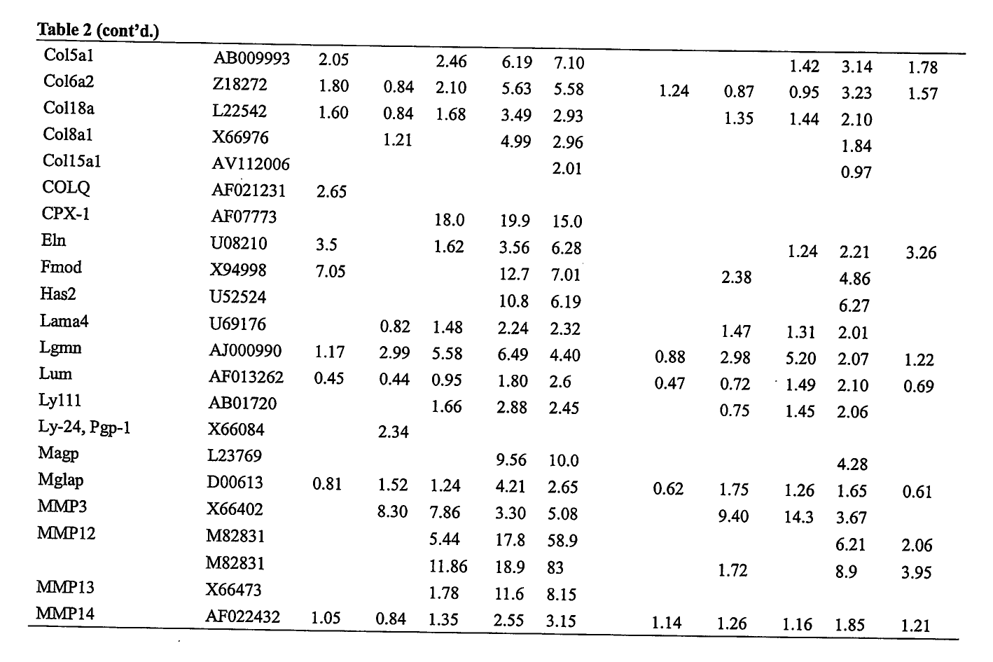

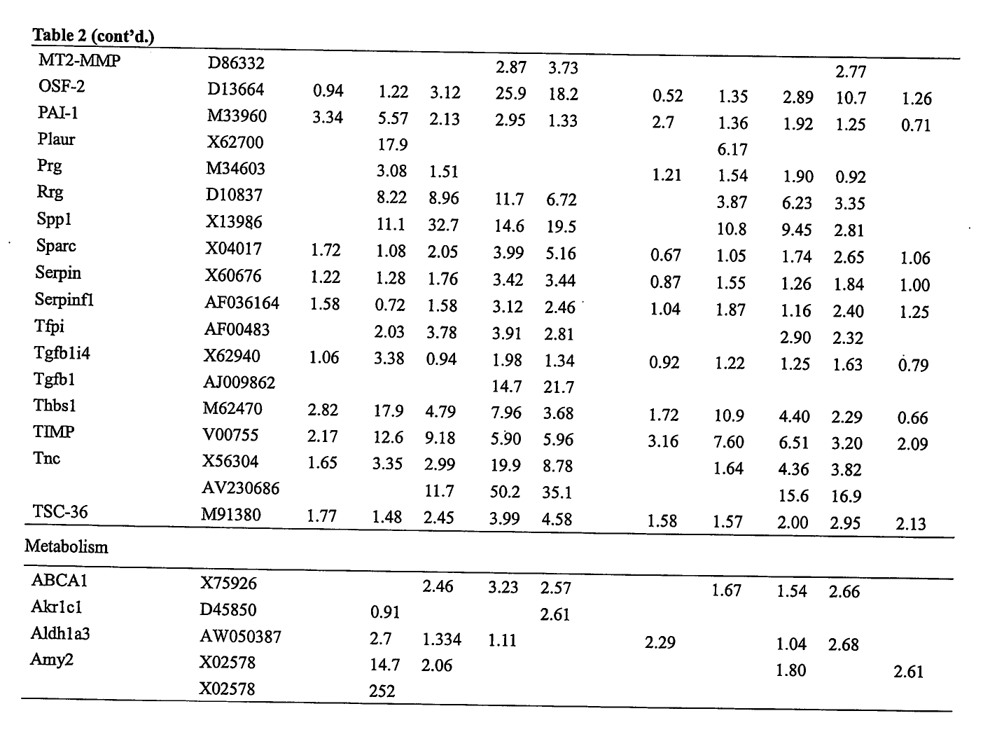

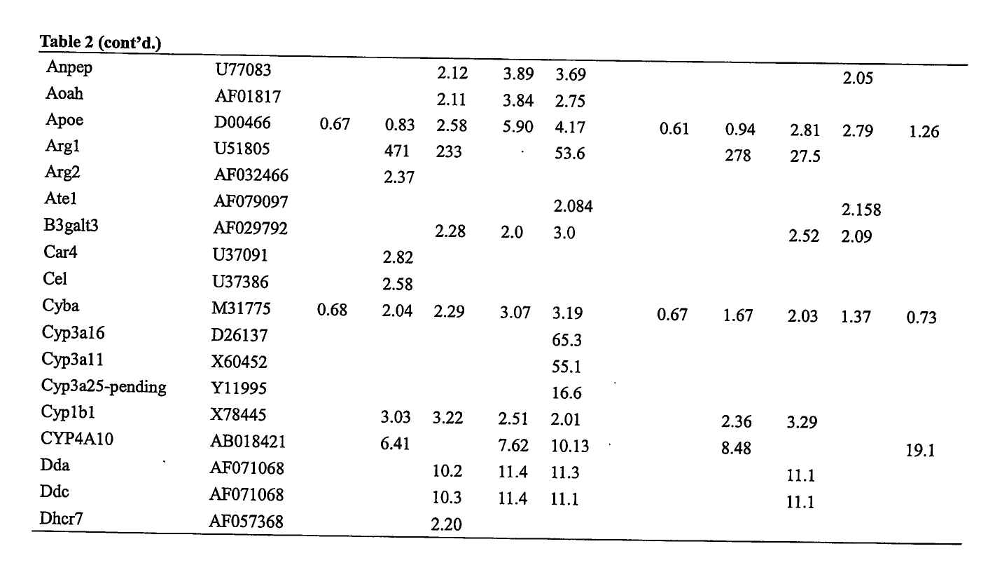

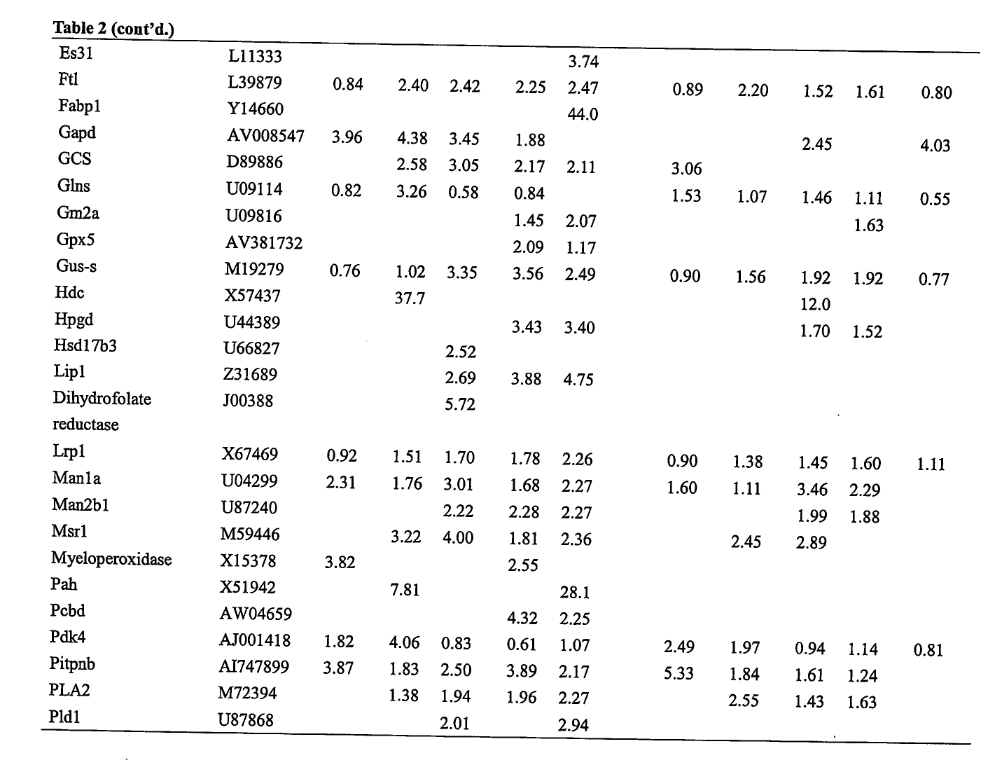

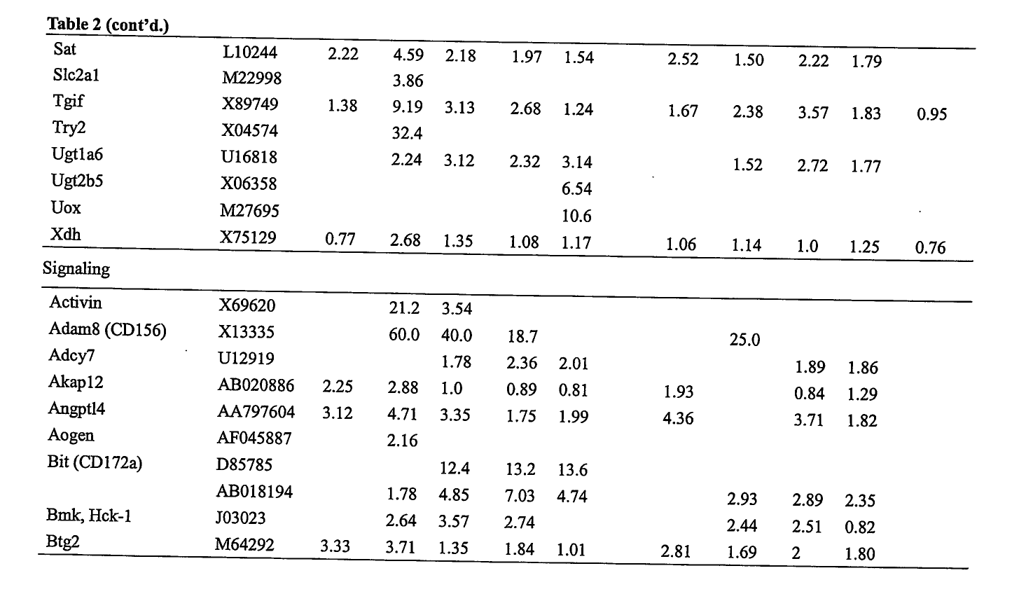

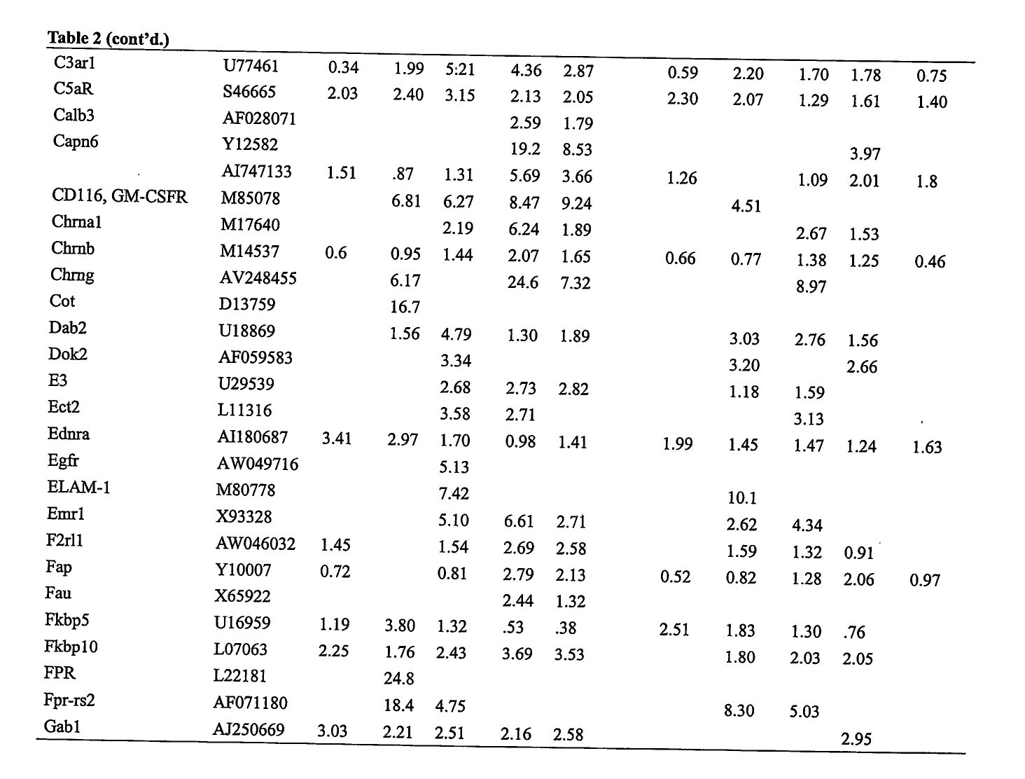

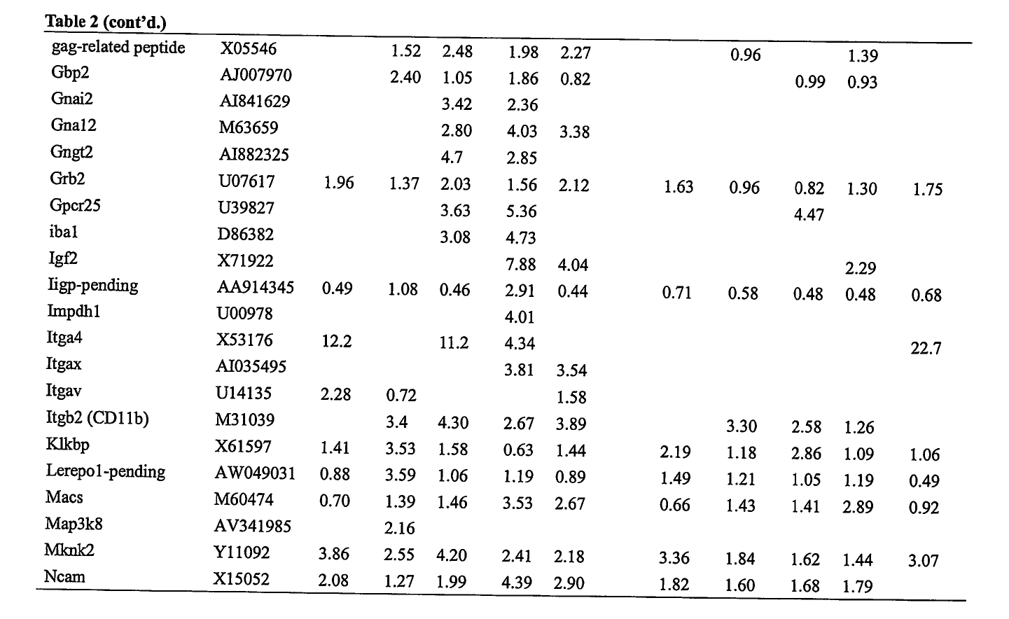

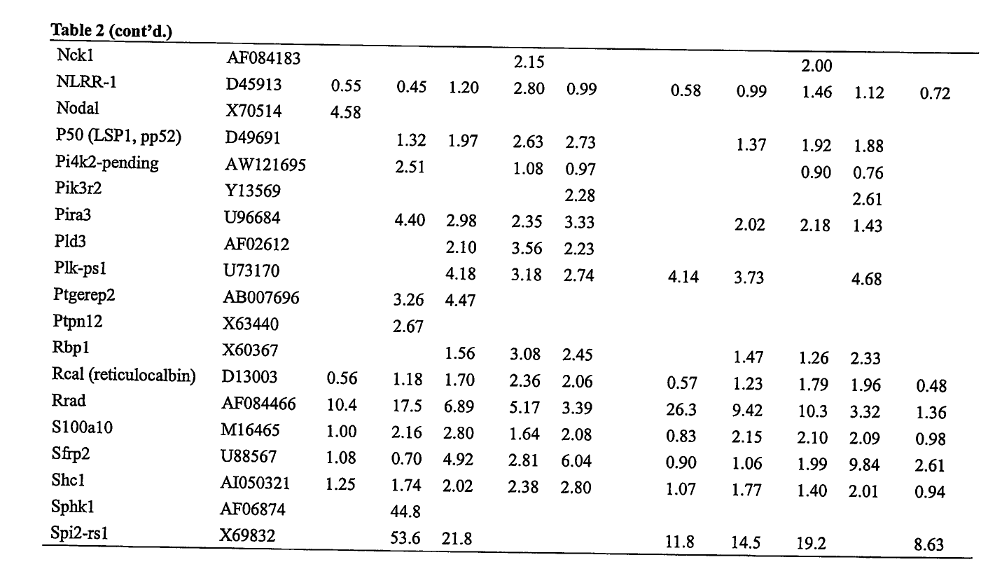

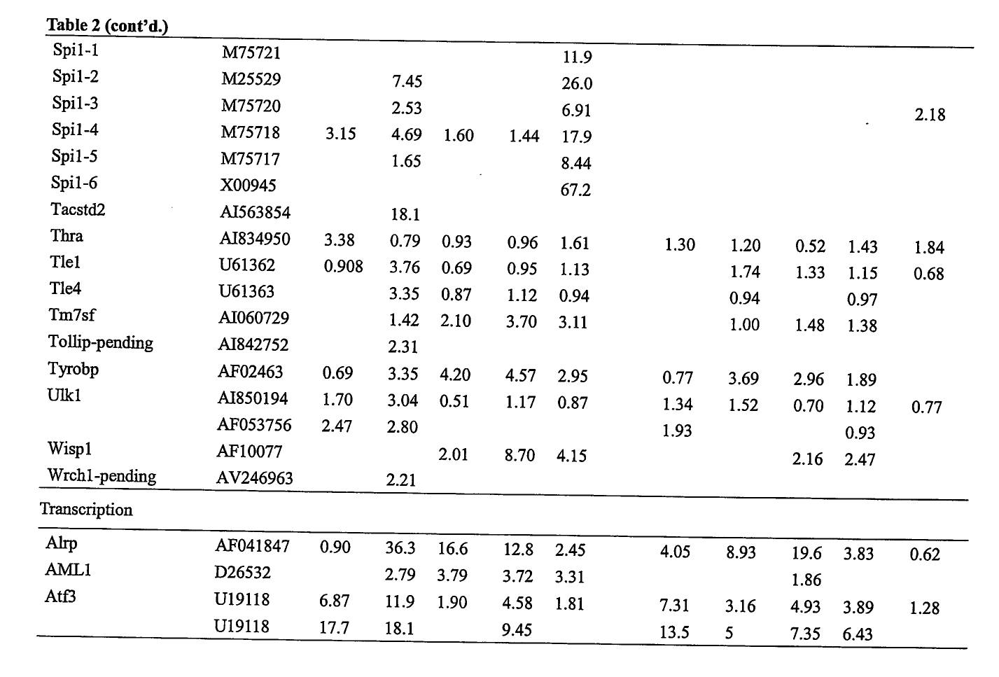

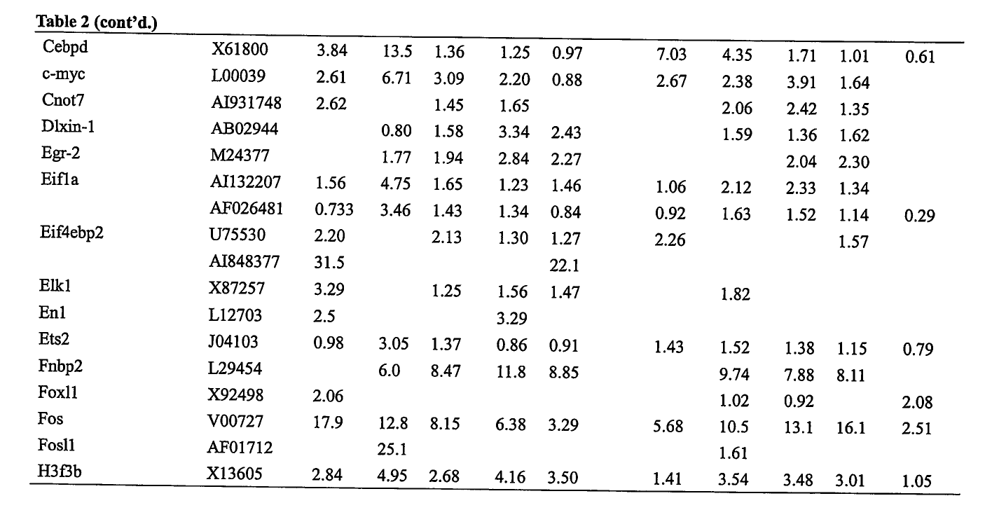

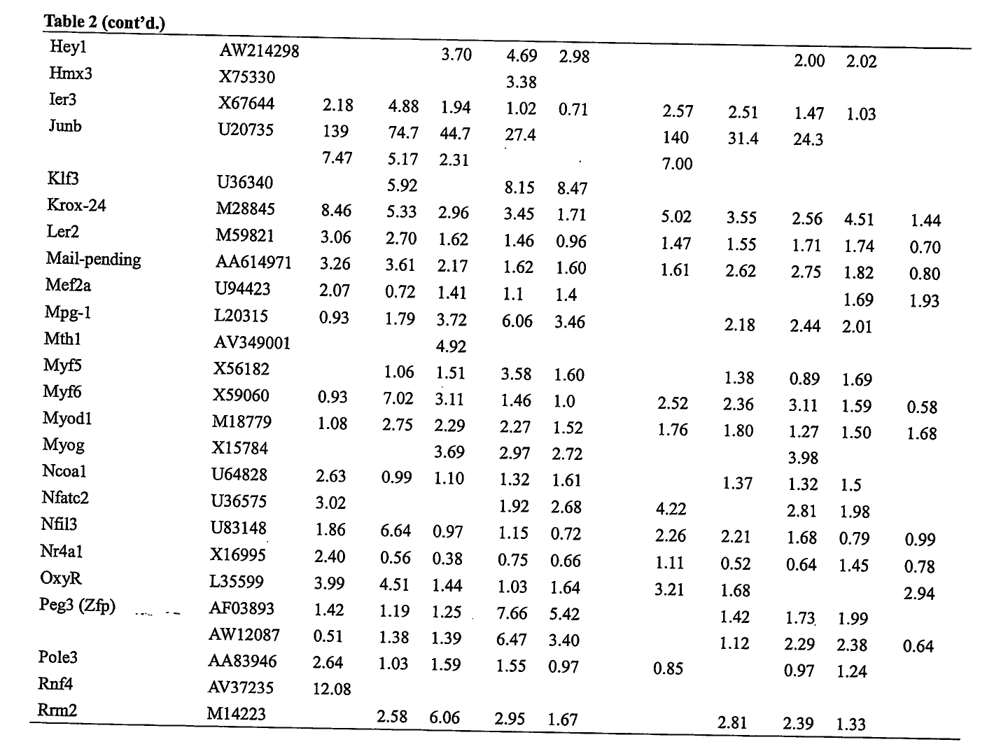

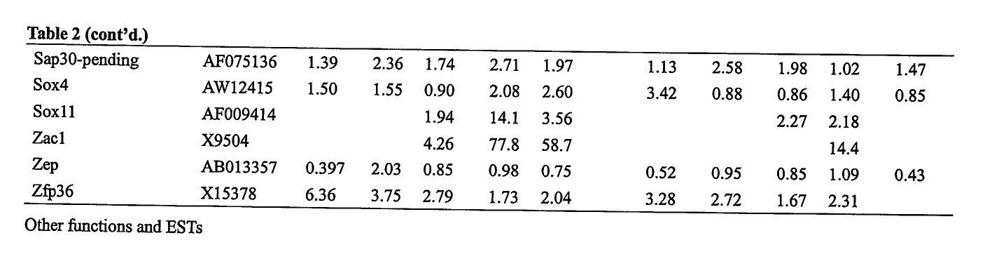

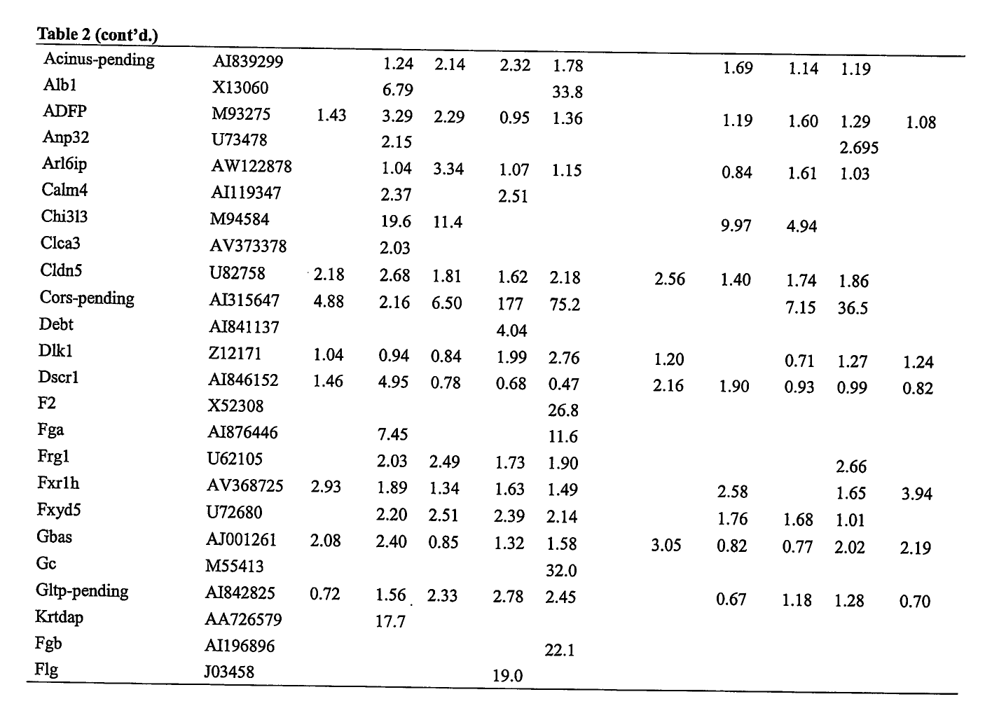

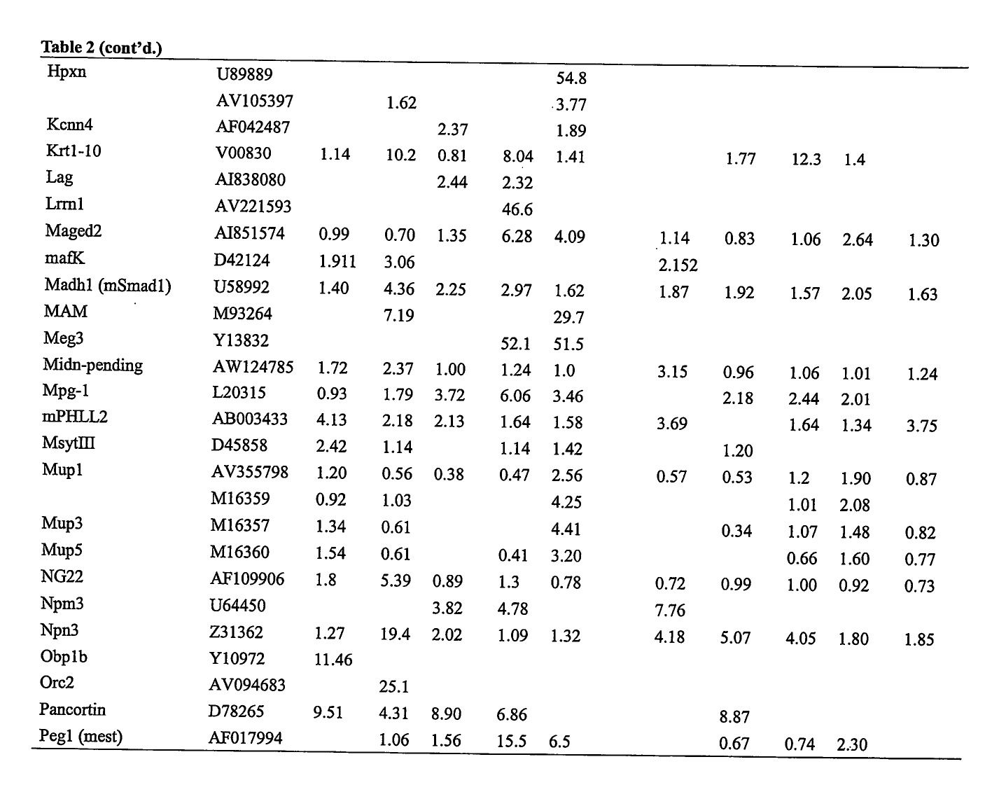

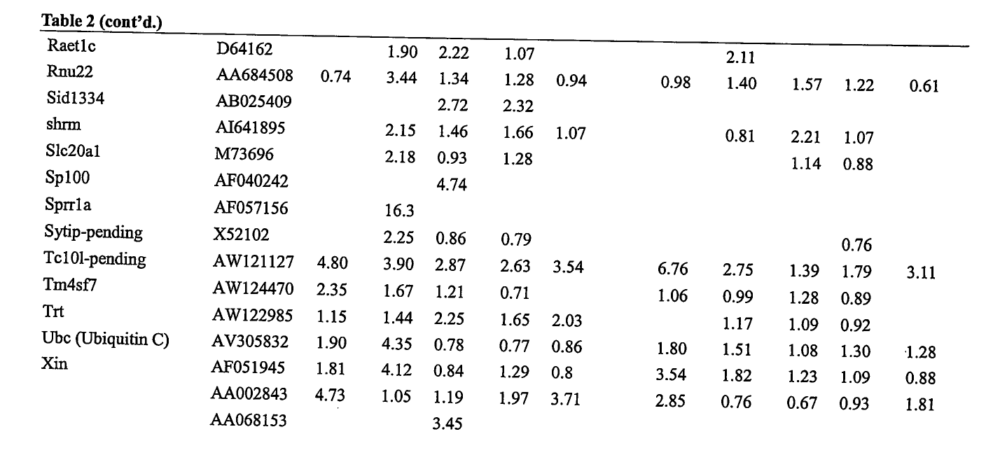

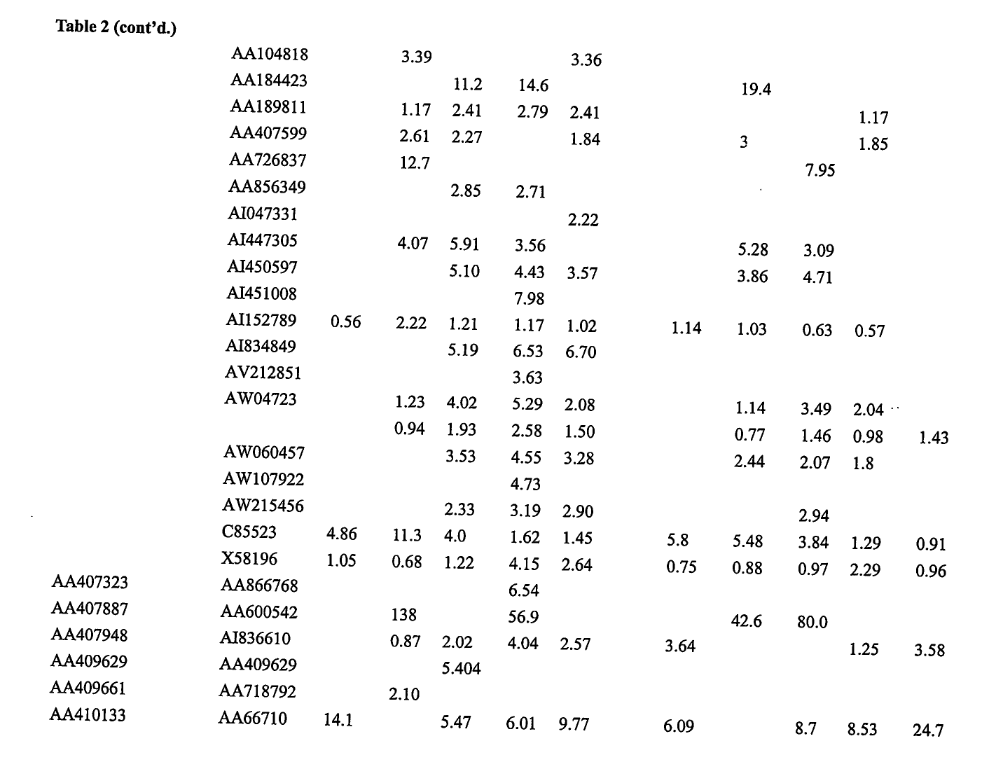

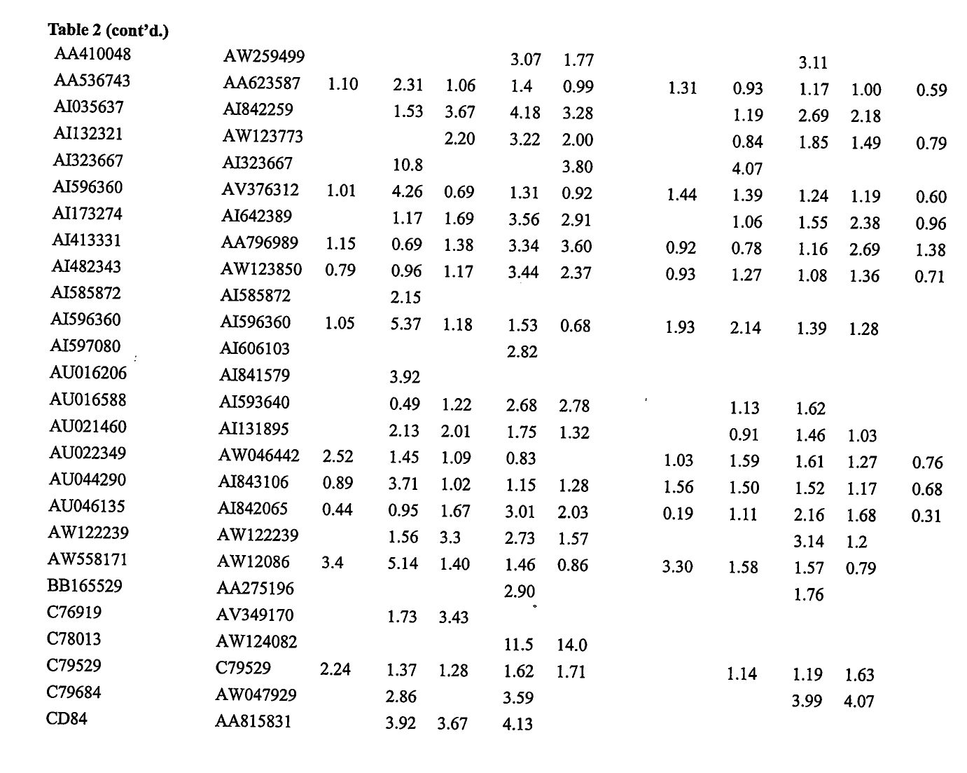

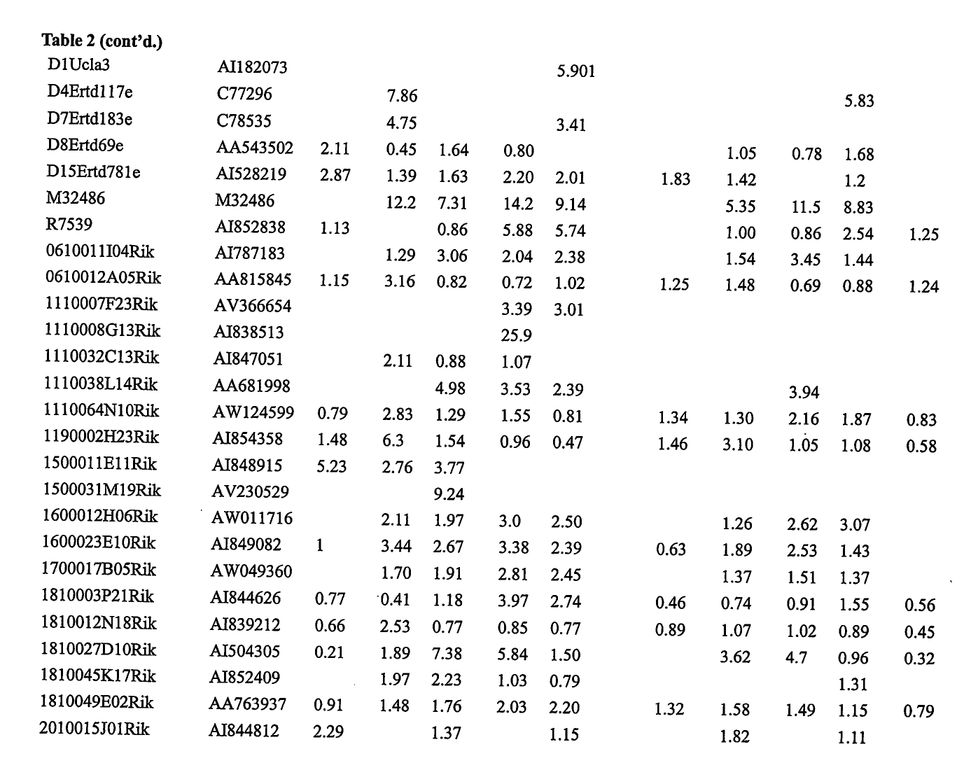

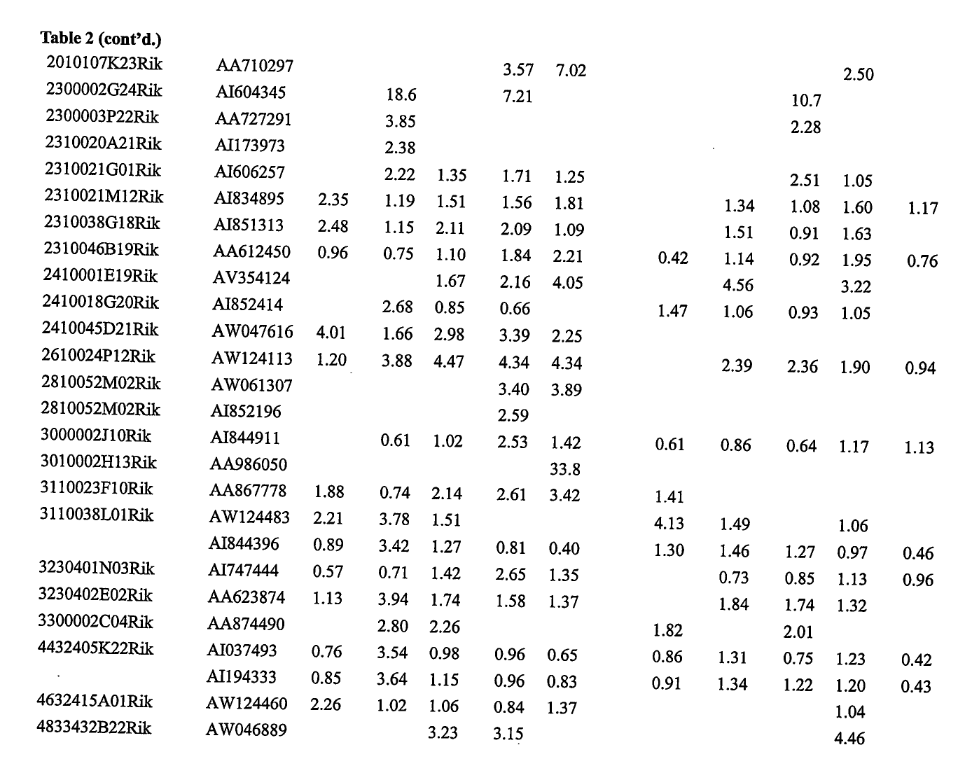

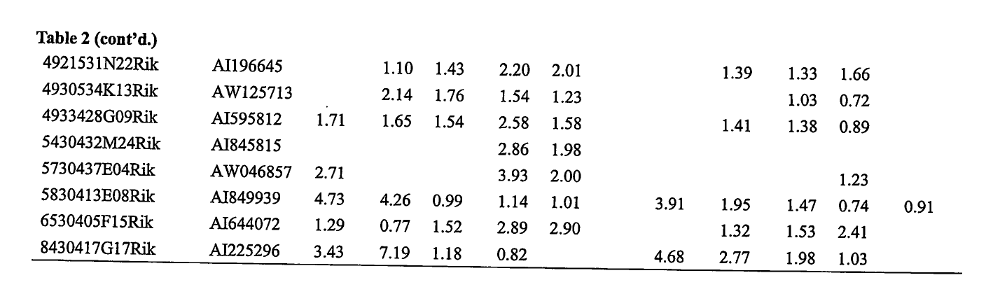

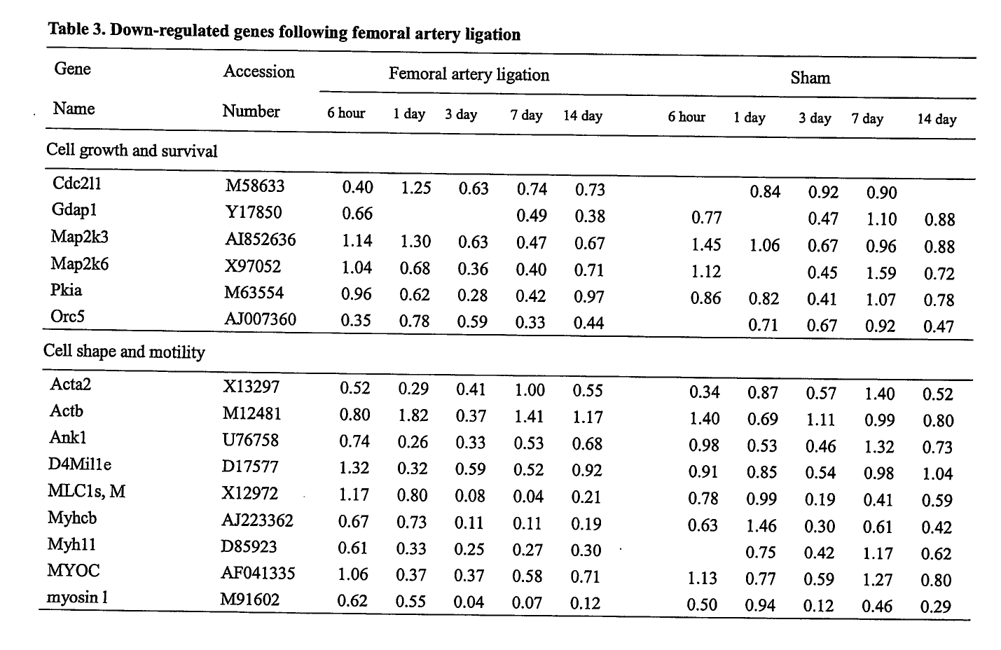

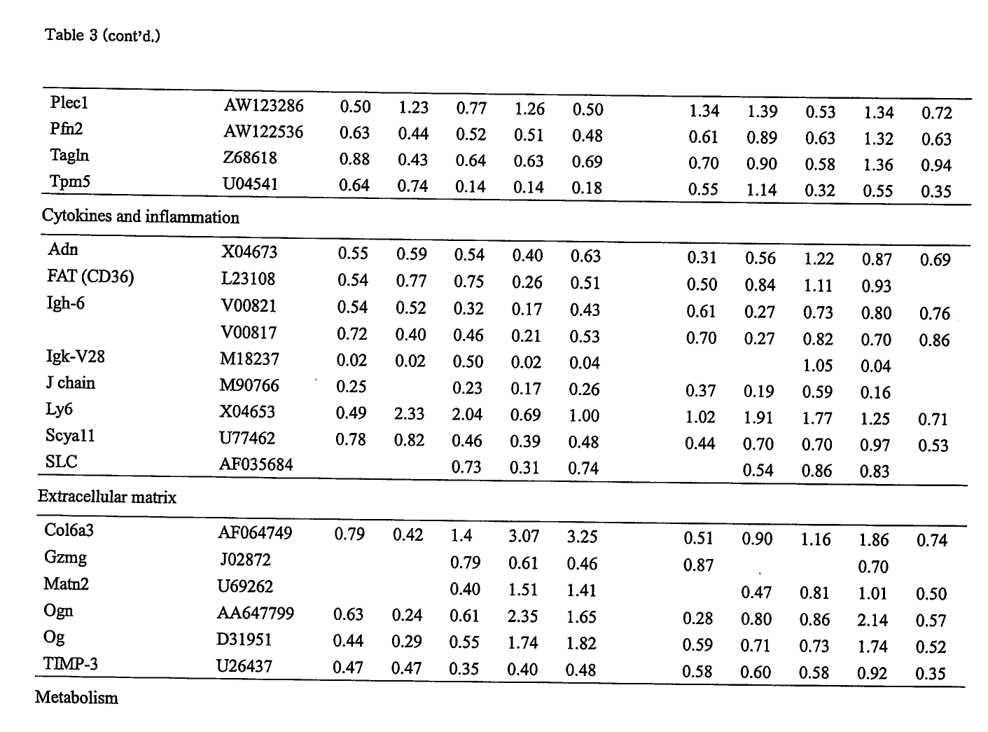

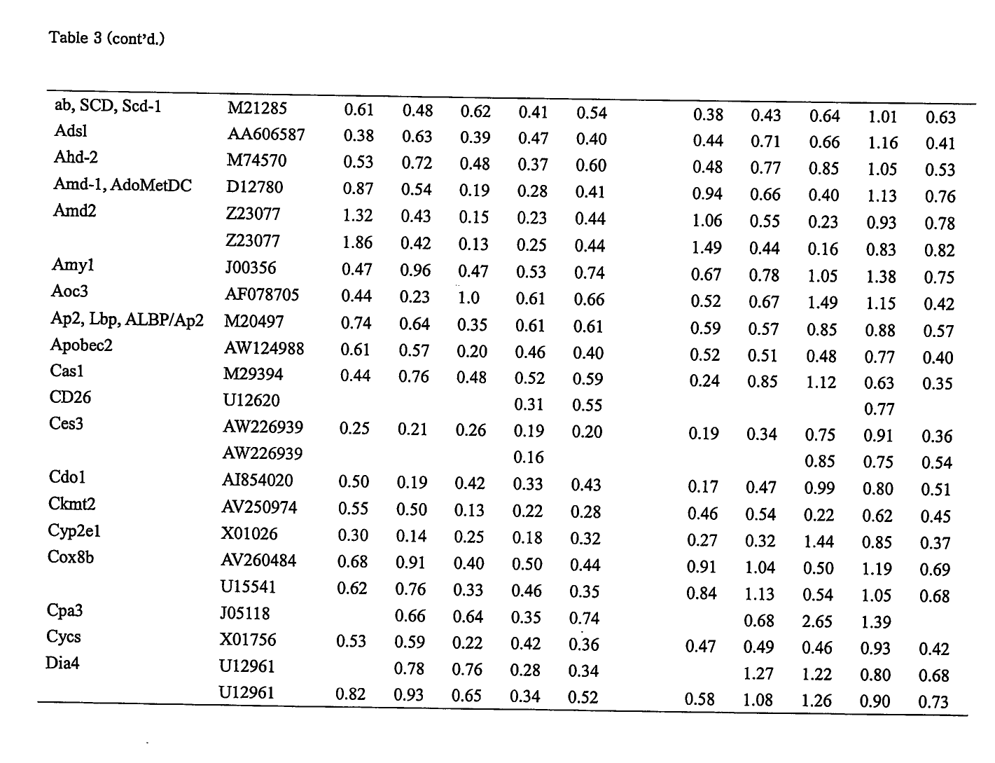

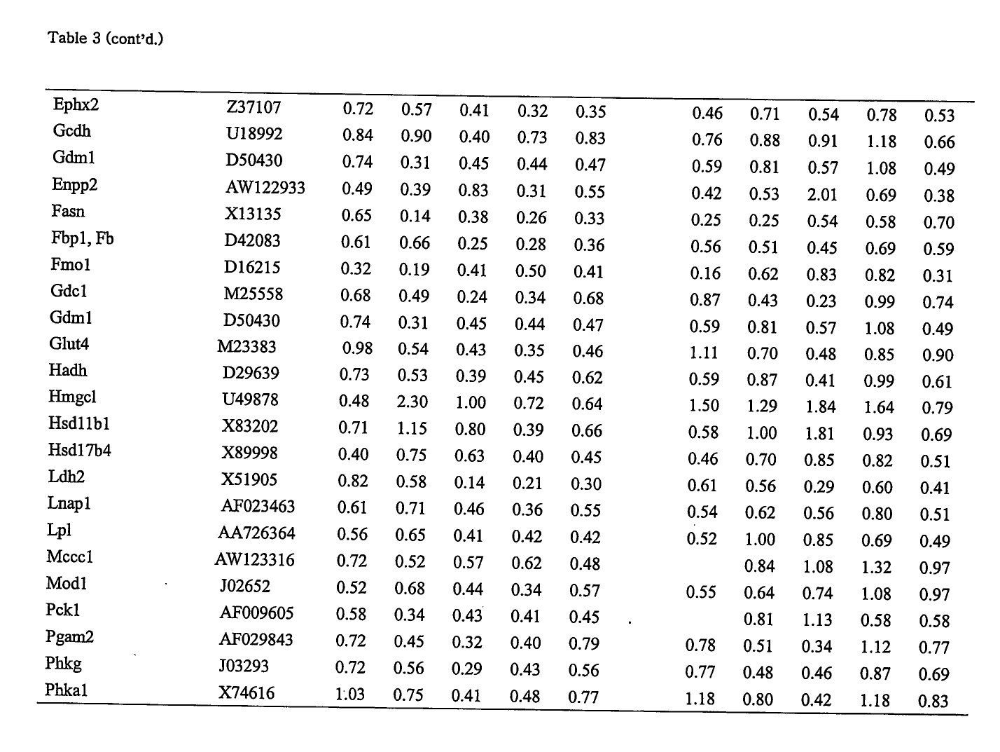

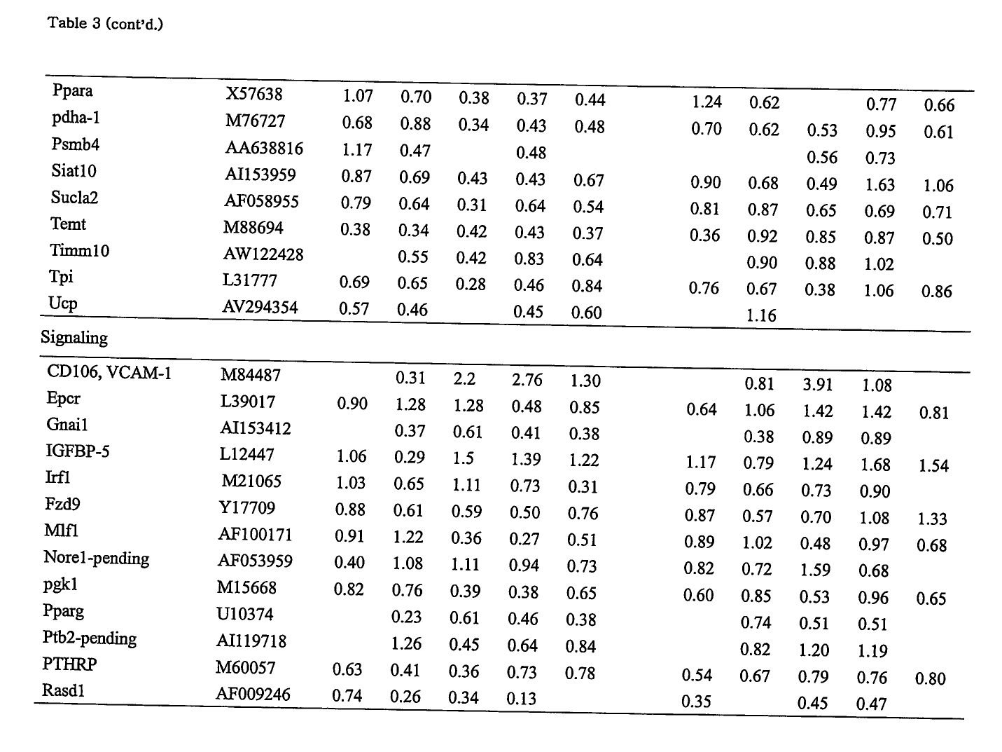

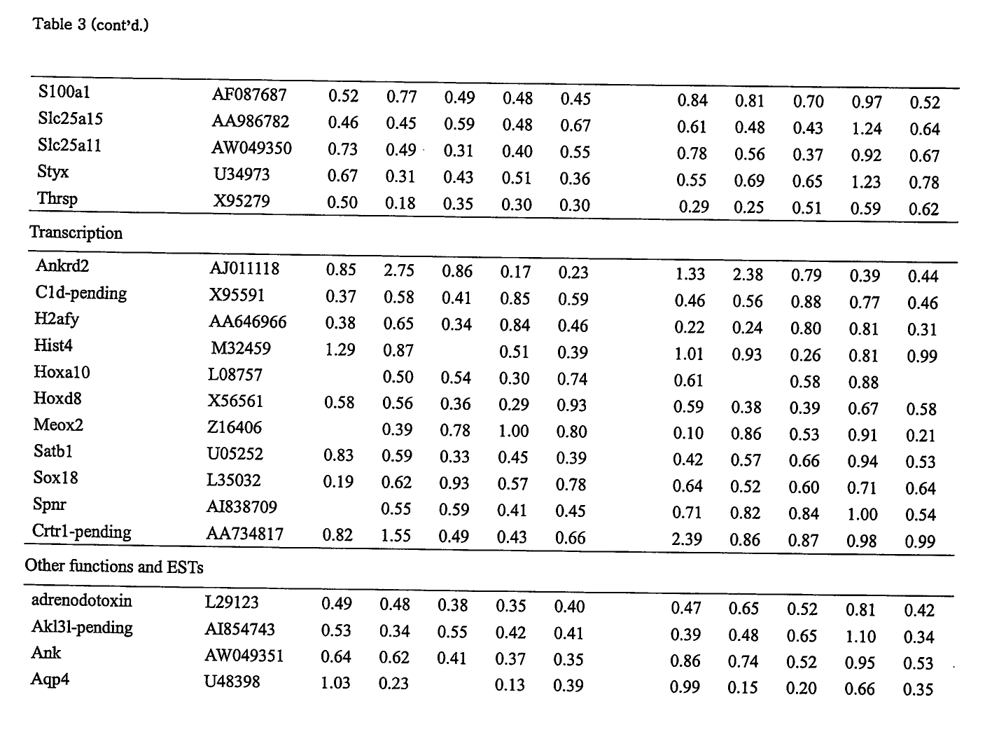

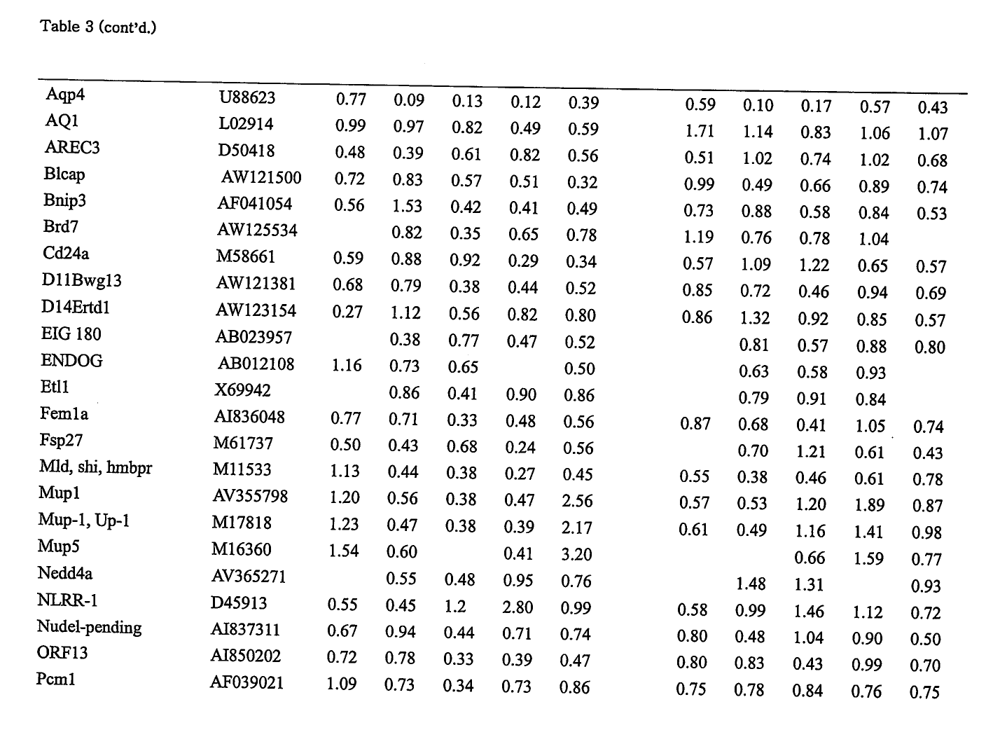

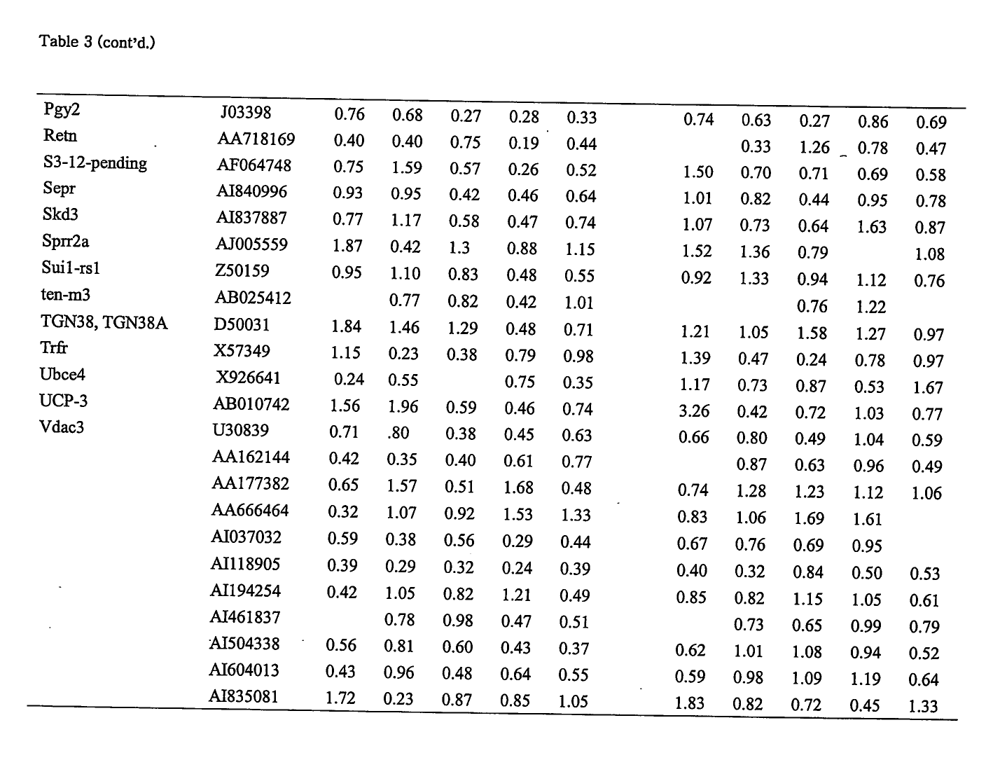

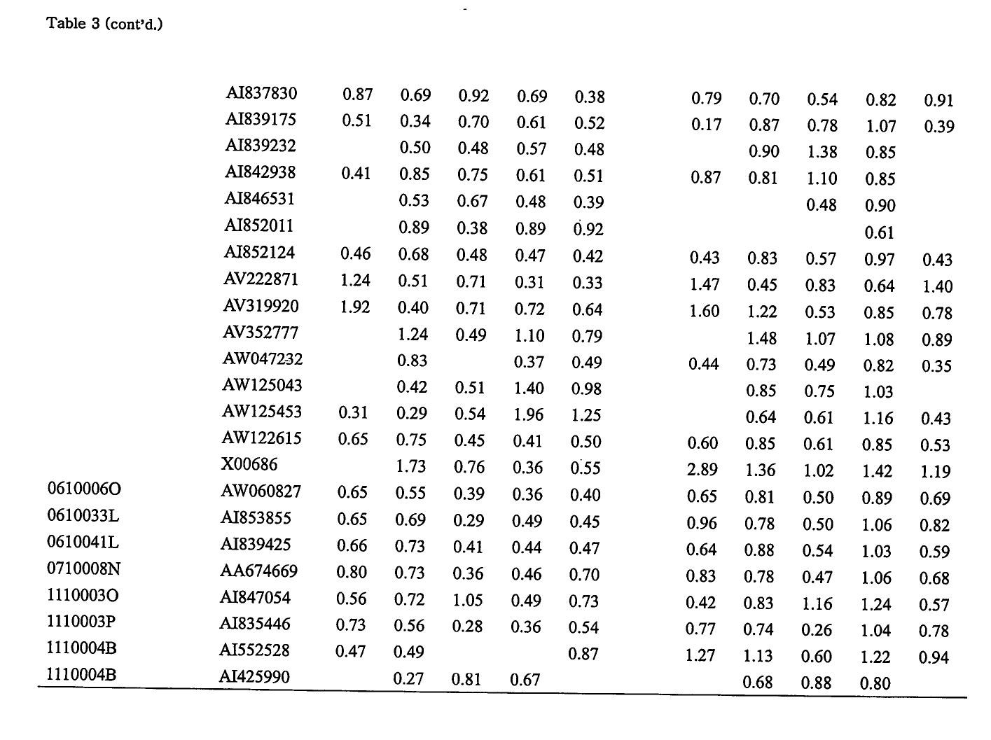

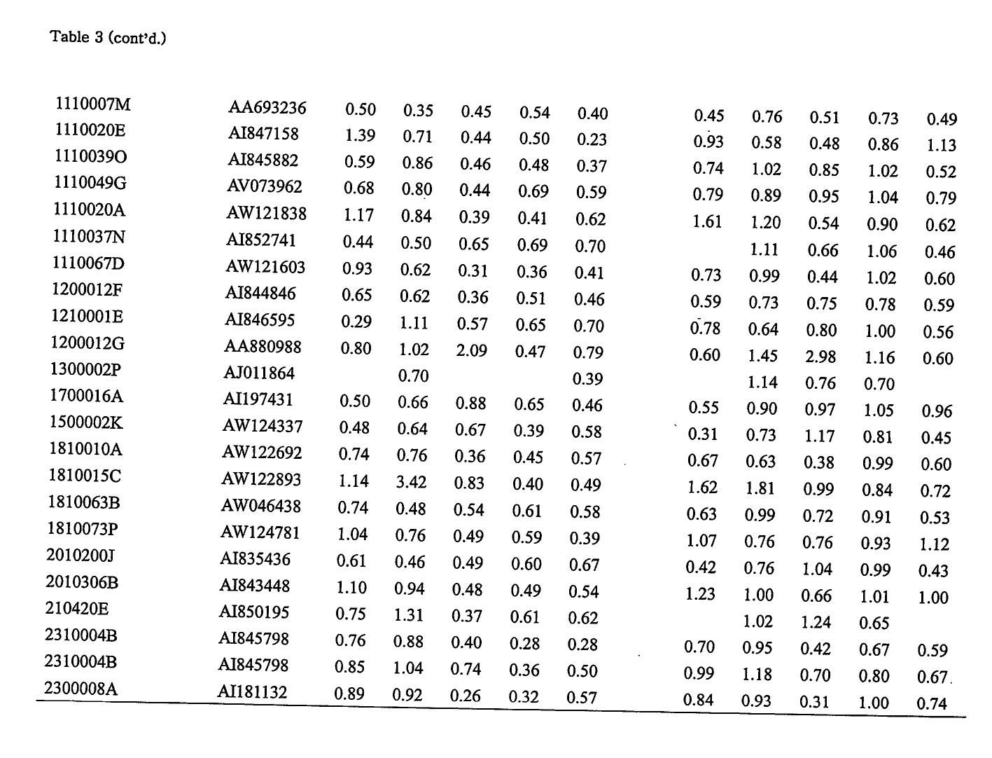

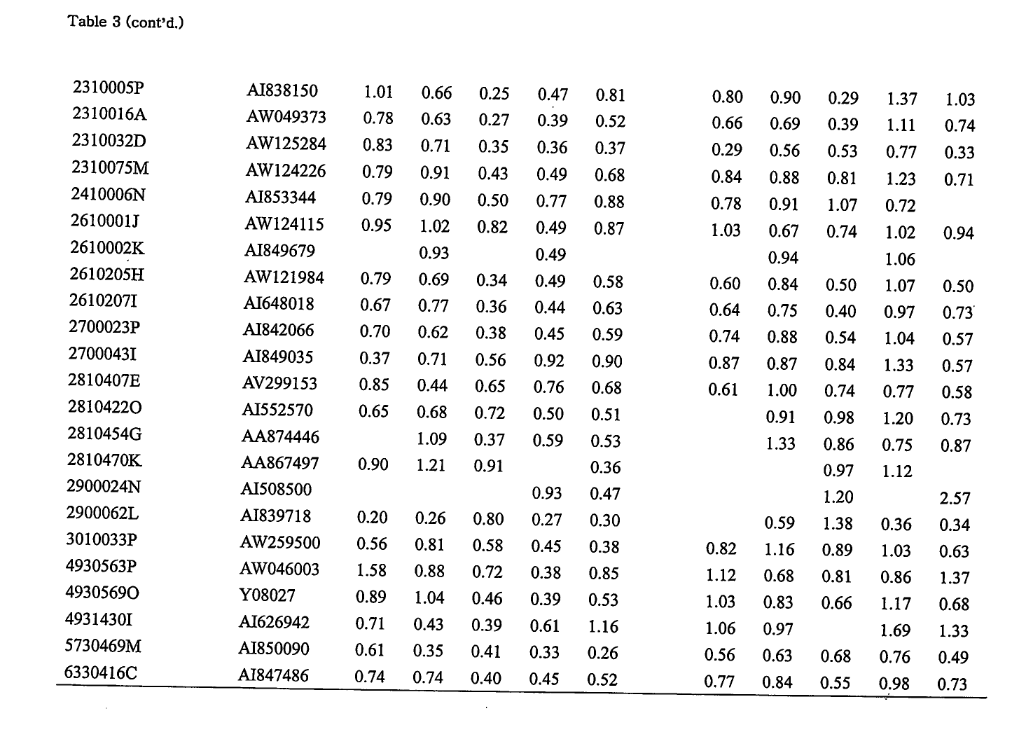

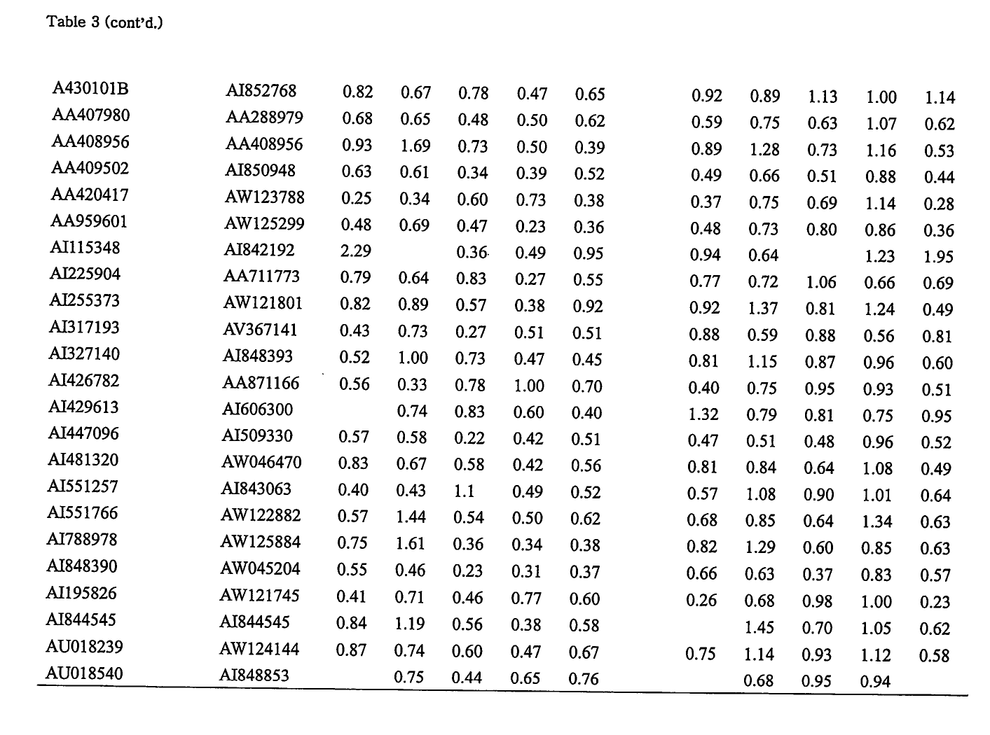

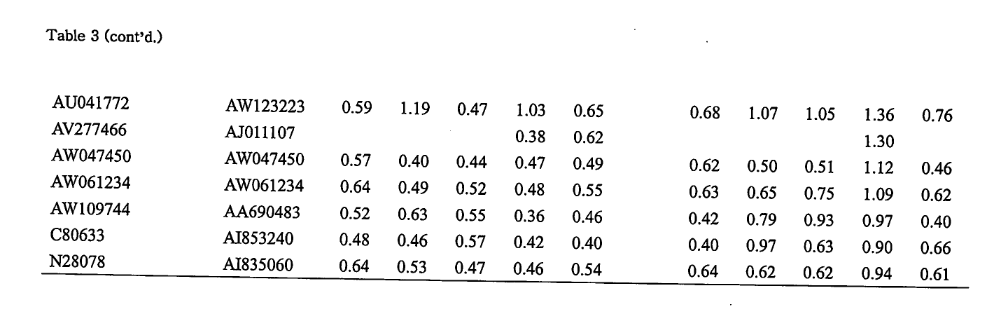

This application claims priority to U.S. Provisional Application Ser. No. 60/432,005, filed Dec. 10, 2002, the contents of which are hereby incorporated by reference in their entirety.

### FIELD OF THE INVENTION

The invention provides compositions and methods for the identification and isolation of genetic elements related to angiogenesis and to the creation and use of arrays containing isolated genetic elements.

### BACKGROUND OF THE INVENTION

Coronary artery disease and peripheral vascular disease are endemic in Western society. In these diseases the arteries that supply blood to the heart muscle or to the legs become narrowed by deposits of fatty, fibrotic, or calcified material on the inside of the artery. The build up of these deposits is called atherosclerosis. Atherosclerosis reduces the blood flow to the muscle of the heart or legs, which starves the muscle of oxygen, leading to either/or angina pectoris (chest pain), myocardial infarction (heart attack), and congestive heart failure, as the disease involves arteries supplying the heart, or pain in the leg (claudication) or leg ulcers if the disease involves arteries supplying the leg.

The body has natural mechanisms whereby new blood vessels, known as collaterals, grow to bypass arterial blockages, although these collaterals rarely are sufficient to restore blood flow to normal. Small narrow collateral blood vessels normally are present, connecting with the large blood vessels that carry the bulk of blood flow, but are too narrow to carry much blood flow under normal conditions. However, after the large vessels to which the collaterals connect become obstructed with atherosclerotic plaque, the collaterals can enlarge so that they are capable of delivering blood to the tissues originally supplied by the now obstructed vessel.

The use of recombinant genes or growth-factors to enhance myocardial collateral blood vessel function represents a new approach to the treatment of cardiovascular disease. Kornowski, R., et al., “Delivery strategies for therapeutic myocardial angiogenesis”, Circulation 2000; 101:454-458. Proof of concept has been demonstrated in animal models of myocardial ischemia, and clinical trials are underway. Unger, E. F., et al., “Basic fibroblast growth factor enhances myocardial collateral flow in a canine model”, Am J Physiol 1994; 266:H1588-1595; Banai, S. et al., “Angiogenic-induced enhancement of collateral blood flow to ischemic myocardium by vascular endothelial growth factor in dogs”, Circulation 1994; 83-2189; Lazarous, D. F., et al., “Effect of chronic systemic administration of basic fibroblast growth factor on collateral development in the canine heart”, Circulation 1995; 91:145-153; Lazarous, D. F., et al., “Comparative effects of basic development and the arterial response to injury”, Circulation 1996; 94:1074-1082; Giordano, F. J., et al., “Intracoronary gene transfer of fibroblast growth factor-5 increases blood flow and contractile function in an ischemic region of the heart”, Nature Med 1996; 2:534-9.

Despite the promising hope for therapeutic angiogenesis as a new modality to treat patients with coronary artery disease, it is apparent that new strategies for optimally promoting clinically relevant therapeutic angiogenic responses are greatly to be desired. In particular, Moreover, new and improved angiogenesis strategies cause functionally that can cause relevant improvement in blood flow to an affected tissue are greatly desirable.

### SUMMARY OF THE INVENTION

The present invention overcomes the problems and disadvantages associated with current strategies and designs and provides kits, compositions and methods for angiotyping” individual patients to predict the likelihood of whether a given individual will develop good vs. poor collaterals naturally.

Several animal studies suggest that factors may exist that interfere with collateral growth—these include diabetes and hypercholesterolemia. There are subgroups of patients with coronary artery disease who have poor collaterals, and others who have excellent collaterals. Impaired collateral development occurring in response to arterial obstructive disease, or in response to angiogenesis interventions, is determined to a large extent by genetic factors (such as specific genetic polymorphisms), and/or by epigenetic factors (such as DNA methylation patterns) that alter the expression of genes encoding angiogenesis factors. Because of the marked individual variability that exists in the capacity to develop collaterals, and because such individual variability is based in large part on genetic and epigenetic differences among patients, it is important to be able to diagnose whether 1) a given patient is likely to develop good vs. poor collaterals naturally, and 2) a given patient is likely to respond to a specific therapeutic angiogenesis strategy. Because of these individual differences, angiogenesis treatment can ultimately be tailored to the individual patient. Therefore, the present invention permits, through DNA and/or protein expression profiling using DNA chips or similar technology, diagnostic “angiotyping” of individual patients to predict the likelihood of whether a given individual will develop good vs. poor collaterals naturally, or in response to specific angiogenesis therapy.

One embodiment of the invention is directed to methods for “angiotyping” individual patients to predict the likelihood of whether a given individual will develop good vs. poor collaterals naturally. Accordingly, this can involve obtaining and providing a list of genes involved in collateral development.

Another embodiment of the invention is directed to methods for “angiotyping” individual patients to predict the likelihood of whether a given individual will develop good vs. poor collaterals in response to specific angiogenesis therapy.

Another embodiment of the invention is directed to methods for the detection of good vs. poor collaterals, comprising the detection of single nucleotide polymorphisms (SNPs) of an array of genes that have been determined through our experimental studies as being differentially expressed in tissues in which collaterals are developing in response to arterial occlusion. SNPs are detected using microchips or similar technology assaying for all, or most, of the genes determined to play a role in collateral development. The presence of a predisposition to develop poor vs. good collaterals is indicated by the presence of SNPs involving one or more of the genes we have determined are involved in those processes leading to enhanced collateral development.

Another embodiment of the invention is directed to methods for the detection of good vs. poor collaterals, comprises the detection of alterations of proteins in the blood, for example in peripheral blood mononuclear cells, expressed by the array of genes that have been determined through our experimental studies as being differentially expressed in tissues in which collaterals are developing in response to arterial occlusion. Protein levels will be either higher than normal levels, lower than normal levels, or the proteins will be post-translationally modified, such as, but not limited to changes in phosphorylation states. The determination of such protein levels/modifications can be by standard assays of individual proteins (ELISA, etc), or by newer methods, such as proteomic analysis. The presence of a predisposition to develop poor vs. good collaterals is indicated by the presence of lower or higher blood levels of proteins that are encoded by one or more of the genes we have determined are involved in those processes leading to enhanced collateral development. The levels of protein can be measured, for example, in the blood fluid and/or in blood cells, such as peripheral blood mononuclear cells (PBMCs).

Another embodiment of the invention is directed to methods for the detection of good vs. poor collaterals, and comprises the detection of DNA methylation patterns involving those genes that have been determined to be differentially expressed in tissues in which collaterals are developing in response to arterial occlusion. The presence of a predisposition to develop poor vs good collaterals is indicated by the presence of DNA methylation patterns that alter gene expression, resulting in lower or higher blood levels of proteins that are encoded by one or more of the genes we have determined are involved in those processes leading to enhanced collateral development.

Another embodiment of the invention is directed to kits suitable for performing genetic microarray analysis for detection, where the kit comprises reagents, such as nucleic acid arrays (gene ships) or PCR primer sets that can detect relevant SNPs of most or all of the genes that have been determined to be involved in those processes leading to enhanced collateral development. The genes may be selected from the group of genes listed in Table 1. The sample may comprise, lymph, venous or arterial blood, and/or vascular tissue of the individual. In one embodiment the polymorphisms are detected using a genetic microarray. In another embodiment the polymorphisms are detected using quantitative PCR.

Another embodiment of the invention is directed to kits for carrying out any of the methods described above.

In specific embodiments the invention provides a method for predicting the likelihood that a subject will develop collaterals, comprising assaying the expression level of at least three in genes in the subject. in a sample obtained from the mammal. The likelihood of collateral development may be predicted by the altered expression of at least three, at least five, at least ten, at least twenty genes, or at least twenty genes in the sample. The altered expression may be increased or decreased expression. Genes having increased and decreased expression are listed in Tables 2 and 3 respectively. The altered expression level may be at least two fold higher or lower than a reference level. The level of gene expression may be determined by assaying the level of protein expression in a sample. In each of these embodiments, the sample may contain blood from the subject and/or may contain blood cells, such as PBMCs, from the subject.

In other embodiments of the invention, there is provided a method for predicting the likelihood that a subject will develop collaterals, comprising detecting the presence of at least three genetic variations in a sample from the patient, where the genetic variations are SNPs or altered DNA methylation patterns. The likelihood of collateral development can be predicted by the presence of genetic variations in at least three, at least five, at least ten, at least twenty genes, or at least twenty genes in the sample. The genes may be selected from the group consisting of the genes listed in Table 1. The method of assay may comprise using a genetic microarray or quantitative PCR, and may be a method to detect DNA methylation patterns and/or to detect single nucleotide polymorphisms.

The invention also provides a kit for carrying out the assays described above, where the assay is to be carried out using a PCR and where the kit comprises a set of primers suitable for amplifying at least three, at least five, at least ten, or at least twenty DNA or RNA sequences corresponding to the genes in Table 1. In another example, there is provided a kit for carrying out the assays described above where the kit comprises a nucleic acid array capable of detecting single nucleotide polymorphisms in a plurality or majority of the genes identified in Table 1.

In another embodiment, the expression level of the genes may be determined by measuring the concentration of the proteins, for example, soluble proteins, encoded by the genes listed in Table 1. The sample from the subject may be blood, and/or lymph. The level of protein expressions may, for example, be determined by ELISA.

The invention also provides methods for promoting collateral formation in a subject, by administering to the subject a composition that decreases expression of at least one gene identified in Table 2 and/or that increases expression of at least one gene identified in Table 3. The composition may contain an antisense oligonucleotide, an siRNA molecule, an RNAi molecule, an oligonucleotide that binds to mRNA to form a triplex, or a DNA molecule that is transcribed in the subject to produce an antisense oligonucleotide, an siRNA molecule, an RNAi, or an oligonucleotide that binds to mRNA to form a triplex. The composition may contain an antibody or a soluble protein receptor, for example, a human antibody or a human soluble protein receptor, that binds to a protein that inhibits collateral formation in the subject. The composition may comprise a protein that is administered to supplement the loss of a protein encoded by a gene identified in Table 3.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF THE FIGURES

Table 1 lists the genes whose expression was detectably altered during the development of collaterals.

Table 2 lists the genes whose expression was increased during the development of collaterals, and also shows the time course of the changes in gene expression.

Table 3 lists the genes whose expression was decreased during the development of collaterals, and also shows the time course of the changes in gene expression

### DETAILED DESCRIPTION OF THE INVENTION

The present invention provides kits, compositions and methods for angiotyping individual patients and for predicting the likelihood of whether a given individual will develop good vs. poor collaterals, either naturally or in response to specific angiogenesis therapy. Specifically, those genes that have altered expression levels during the development of collaterals have been identified, and the changes in gene expression have been quantified. By measuring changes in gene expression, the risk of whether a given individual will develop good vs. poor collaterals naturally or in response to specific angiogenesis therapy can be determined. Moreover, the relative changes in gene expression at different time points during the collateral development process have been measured, and these measurements allow additional insight into the progress and development of collaterals.

Because differential expression of genes is involved in collateral development, changes in the degree of expression, or in the length of time during which they are differentially expressed, lead to different degrees of collateral development. In the context of coronary artery disease and peripheral vascular disease, differing degrees of collateral development can cause some individuals to have minimal symptoms in association with atherosclerotic arterial obstructive disease, and other individuals to have severe symptoms. Changes in the degree of gene expression, or in the length of time during which the genes are differentially expressed, are caused by polymorphisms either in the coding region of the gene or in the regulatory components of the gene. Alternatively, these changes can be caused by “epigenetic alterations,” such as, but not limited to, changes in DNA methylation patterns. By correlating changes in gene expression with collateral development, the present invention identifies those genes in which polymorphisms or altered DNA methylation patterns can convey susceptibility to the development of either poor vs good collateral development.

The identification of genes that are involved in collateral development allows those genes having changed degree or duration of expression, caused in part by polymorphisms of the gene or alterations in DNA methylation patterns, to be used as targets to identify genetic abnormalities conveying altered capacities to develop collaterals. Identification of polymorphisms or alterations in DNA methylation patterns allows prediction of the risk for poor collateral development in patients prior to the performance of angioplasty procedures or the initiation of angiogenesis therapy. Once pre-procedure risk prediction is possible, this will importantly influence how a patient is treated. Some patients deemed to be resistant to the development of collaterals might be offered bypass surgery or angioplasty. Others might forego angiogenesis therapy and be treated aggressively with brachytherapy (intravascular radiation). Accordingly, the present invention provides new and improved methods for “angiotyping” individual patients to predict the likelihood of whether a given individual will develop good vs poor collaterals naturally or in response to specific angiogenesis therapy.

Moreover, identification of the genes that are abnormally expressed by an individual patient because of either a SNP or an altered DNA methylation pattern, provides new methods for ameliorating or treating the disease by therapy targeted to a specific set or subset of those genes with altered expression. Because different polymorphisms and DNA methylation patterns play a role in the development of collaterals in different patients, the invention allows identification of specific abnormalities that may be characteristic to a specific patient. The invention therefore allows for greater specificity of treatment. A regime that may be efficacious in one patient with a specific polymorphism profile may not be effective in a second patient with a different polymorphism profile. Such profiling also allows treatment to be individualized so that unnecessary side effects of a treatment strategy that would not be effective for a specific patient can be avoided.

Specifically, approximately five hundred and seventy five genes are identified whose expression changes during the course of collateral development. Since the differential expression of these genes is involved in collateral development, changes in the degree of expression, or in the length of time during which they are differentially expressed, leads to altered capacity to develop collaterals.

Changes in the degree of gene expression, or in the length of time during which the genes are differentially expressed, can be caused by polymorphisms in the gene or in the regulatory components of the gene. Such polymorphisms, conveying an increased risk of disease development, have already been identified for several genes associated with several diseases. This invention, therefore, identifies those genes in which polymorphisms can convey susceptibility to poor vs good collateral development. Similar predictions can derive from altered gene expression caused by altered DNA methylation patterns, which can relate to specific SNPs, or regulate gene expression independently of SNPs. Subsequent reference, therefore, to prediction of good vs poor collateral development, relate to polymorphisms of the genes identified by this invention, or of their regulatory units, or to altered DNA methylation patterns which in turn alter gene expression.

The change in expression of certain of the identified genes is predictive of the capacity to develop poor vs. good collaterals. By identifying 575 genes whose expression changes during collateral development, the inventors recognize that analysis of greater numbers of polymorphisms or DNA methylation patterns of those genes leads to a greater ability to predict the capacity to develop collaterals. The role played by these genes in collateral development means that an ability to manipulate the expression of those genes permits improved treatment of arterial obstructive disease . The skilled artisan will recognize that methods to enhance or decrease gene expression are known in the art. For example, methods to enhance collaterals may include gene therapy to increase the expression of genes down-regulated during collateral development. Such gene therapy can be carried out using methods that are known in the art and can used, for example, viral and/or non-viral vectors to deliver nucleic acids that encode and permit expression of a desired gene. Conversely, methods of decreasing expression and/or activity of a desired gene are well known in the art and include, for example, antisense RNA, and RNAi/siRNA methods. Treatment may also include methods to decrease the expression of genes up-regulated during collateral development.

Identification of genes involved in collateral development also permits identification of proteins that affect the development of collaterals. This in turn makes possible the use of methods to expression of these proteins or alter their metabolism. Methods to alter the effect of expressed proteins include, but are not limited to, the use of specific antibodies or antibody fragments that bind the identified proteins, specific receptors and soluble receptor fragments that bind the identified protein, or other ligands or small molecules that inhibit the identified protein from affecting its physiological target and exerting its metabolic and biologic effects. In addition, those proteins that are down-regulated during the course of collateral development may be supplemented exogenously to ameliorate their decreased synthesis.

Different polymorphisms and DNA methylation patterns may play a role in collateral development in different patients. Accordingly, the present invention makes possible an identification of specific abnormalities that are characteristic of a specific patient (“angiotyping”), which allows for greater specificity of treatment. A regime that may be efficacious in one patient with a specific polymorphism profile may not be effective in a second patient with a different polymorphism profile. Such a profiling also allows treatment to be individualized so that unnecessary side effects of a treatment strategy that would not be effective for a specific patient can be avoided.

Elucidation of Changes in Gene Expression in Collateral Development

The inventors have identified the genes that undergo changes in expression during collateral development. Those genes are listed in Table 1. Those genes that exhibit increased and decreased expression during collateral development are shown in Tables 2 and 3 respectively, together with measurements of the teinporal changes in expression. The inventors have carried out this analysis using nucleic acid array analysis of murine adductor muscles as described in more detail below. The skilled artisan will recognize, however, that additional methods for measuring gene expression are well known in the art.

The mouse is a widely accepted model for the human for vascular studies, and results obtained in the mouse are considered highly predictive of results in humans. Accordingly, it is expected that the changes in gene expression in humans during collateral development will be similar to or essentially the same as those observed in the mouse. Exaggerated changes in the degree of expression in these genes, or in the length of time during which the genes are differentially expressed, will predispose to good vs poor collaterals. Such exaggerated changes are usually caused by polymorphisms in the gene or in the regulatory components. of the gene, and therefore the mouse genes identified as being differentially regulated during the angiogenic process will be homologous to the human genes in which such polymorphisms will be found to convey the ability to form good vs. poor collaterals. Moreover, both mouse and human homologues are known for each of the genes described in Table 1, demonstrating further that the results obtained in the mouse studies will be highly predictive of results obtained in humans.

The genes for which, in a given patient, either SNPs or altered DNA methylation patterns are observed, and that are associated with collateral development, also serve as the target for therapeutic interventions. Thus, those genes upregulated during the collateral development can be targeted by therapy designed to decrease gene expression or function of the proteins encoded by these genes; and those genes down-regulated during collateral development can be targeted by therapy designed to increase gene expression or function of the proteins encoded by these genes.

Changes in gene expression in the mouse ischemic hindlimb during experimentally induced collateral development have been studied, a model commonly accepted as a reasonable animal model simulating collateral development as it occurs in humans. Sample and control mouse hindlimb tissues were obtained, RNA was prepared from the tissues, labeled cRNA generated from it and analyzed using an Affymetrix GeneChip® mouse Genome. Sample and control tissues were compared and those genes that experienced significant changes in gene expression were identified. For the purposes of this study, a two fold increase or decrease in gene expression was deemed significant, although the skilled worker will recognize that under certain circumstances smaller changes in gene expression may also be significant. Corresponding human genes for each of the genes determined to have a significant change in expression were identified.

Although about 575 genes have been shown to have altered expression in collateral development (Table 1), it is possible to reliably predict good vs poor collateral development by analyzing a subset of a few of these genes. In embodiments of the present invention at least five, ten, fifteen, twenty or fifty genes may be studied or, if desired, all or most of the genes listed in Table 1 can be studied. These genes also can be analyzed for polymorphisms or altered DNA methylation patterns that alter gene expression. All of the genes can be analyzed initially, but reliable predictions can be made by analyzing a subset of these genes that contains a few members. In other embodiments, at least five, ten, fifteen, twenty or fifty genes may be studied or, if desired, all or most of the genes listed in Table 1 can be studied, for example, using sequencing, short tandem repeat association studies, single nucleotide polymorphism association studies, etc. In each case, however, it generally is more convenient to study gene expression or polymorphisms in a smaller subset of the genes.

By measuring changes in expression of a set of genes (for example by blood protein analysis or by analysis of proteins in blood cells such as PBMCs), or by identification of polymorphisms or DNA methylation patterns influencing expression of sets of genes, rather than of a single gene, the present invention provides increased statistical confidence that the changes observed are predictive of poor vs. good collateral development, such as by providing reliable risk profiling of an individual. Thus, a change in expression of a single gene, or a single gene polymorphism, may not increase susceptibility to good vs poor collateral development sufficiently to cross the diagnosis threshold. On the other hand, coordinated changes in expression of multiple specified genes, due the presence of multiple polymorphisms and/or DNA methylation patterns, are much more likely to increase the likelihood of poor vs. good collateral development. This is analogous to the situation of an individual have only one risk factor predisposing to atherosclerosis (elevated cholesterol). Risk is increased markedly as the number of risk factors increase (elevated cholesterol plus hypertension, obesity, smoking, diabetes, etc).

Identification of polymorphisms or alterations in DNA methylation patterns allows prediction of the risk for poor collateral development in patients prior to the performance of angioplasty procedures or the initiation of angiogenesis therapy. This pre-procedure risk prediction can be used to influence how the patient is treated. Some patients deemed to be resistant to the development of collaterals might be offered bypass surgery or angioplasty. Others might forego angiogenesis therapy and be treated aggressively with brachytherapy (intravascular radiation). Accordingly, the present invention provides new and improved methods for “angiotyping” individual patients to predict the likelihood of whether a given individual will develop good vs poor collaterals naturally or in response to specific angiogenesis therapy.

Dysregulation of Multiple Genes that Increase Susceptibility to Poor vs Good Collateral Development

Gene polymorphisms and altered DNA methylation patterns that lead to biologically important alterations in the expression of genes that are differentially expressed during collateral development can be measured directly in patient samples. These samples comprise DNA that is most conveniently obtained from peripheral blood, for example from PBMCs. The present inventors used nucleic acid array methods to identify the complete set of genes that exhibit significantly changed expression during the course of the healing response to acute vascular injury. However, other methods for measuring changes in gene expression are well known in the art. For example, levels of proteins can be measured in tissue sample isolates using quantitative immunoassays such as the ELISA. Kits for measuring levels of many proteins using ELISA methods are commercially available from suppliers such as R&D Systems (Minneapolis, Minn.) and ELISA methods also can be developed using well known techniques. See for example Antibodies: A Laboratory Manual (Harlow and Lane Eds. Cold Spring Harbor Press). Antibodies for use in such ELISA methods either are commercially available or may be prepared using well known methods.

Other methods of quantitative analysis of multiple proteins include, for example, proteomics technologies such as isotope coded affinity tag reagents, MALDI TOF/TOF tandem mass spectrometry, and 2D-gel/mass spectrometry technologies. These technologies are commercially available from, for example, Large Scale Proteomics Inc. (Germantown Md.) and Oxford Glycosystems (Oxford UK).

Alternatively, quantitative mRNA amplification methods, such as quantitative RT-PCR, can be used to measure changes in gene expression at the message level. Systems for carrying out these methods also are commercially available, for example the TaqMan system (Roche Molecular System, Alameda, Calif.) and the Light Cycler system (Roche Diagnostics, Indianapolis, Ind.). Methods for devising appropriate primers for use in RT-PCR and related methods are well known in the art. In particular, a number of software packages are commercially available for devising PCR primer sequences.

Nucleic acid arrays offer are a particularly attractive method for studying the expression of multiple genes. In particular, arrays provide a method of simultaneously assaying expression of a large number of genes. Such methods are now well known in the art and commercial systems are available from, for example, Affymetrix (Santa Clara, Calif.), Incyte (Palo Alto, Calif.), Research Genetics (Huntsville, Ala.) and Agilent (Palo Alto, Calif.). See also U.S. Pat. Nos. 5,445,934, 5,700,637, 6,080,585, 6,261,776 which are hereby incorporated by reference in their entirety.

Changes in the degree of gene expression, or in the length of time during which the genes are differentially expressed, can be caused by polymorphisms in the gene or in the regulatory components of the gene. Such polymorphisms, conveying an increased risk of disease development, have already been identified for genes associated with several diseases. The present invention, therefore, identifies those genes in which polymorphisms or altered DNA methylation patterns can convey susceptibility to poor vs good collateral development. It is one object of this invention to identify such polymorphisms by developing a DNA microarray chip containing all those SNPs affecting those genes we have identified as playing a role in collateral development (For example, by using the Affymetrix GeneChip system).

Methods for identifying polymorphisms in genes are well known in the art. See, for example, U.S. Pat. Nos. 6,235,480 and 6,268,146, which are hereby incorporated by reference in their entirety. Once polymorphisms are identified, methods for detecting specific polymorphisms in a gene using nucleic acid arrays are also well known in the art

Thus, in one embodiment, the invention provides methods where SNPs or altered DNA methylation patterns are identified for at least three genes selected from the genes shown in Table 1. In other embodiments of the invention SNPs or altered DNA methylation patterns are determined of at least five genes to determine the likelihood of good vs poor collateral development. In yet further embodiments the number of genes assayed is ten. In yet other embodiments the number of genes assayed is 20 or at least about 20. In still yet other embodiments the number of genes assayed is 50 or at least about 50. Regardless of the number of genes in the subset of analyzed genes, selected from the genes shown in Table 1, the aggregate number of polymorphisms or DNA methylation patterns can then permit prediction of good vs poor collateral development. Similarly, coordinated changes in expression of the genes identified herein also can permit prediction of good vs poor collateral development.

With respect to polymorphisms, as the number of biologically significant polymorphisms increases, so does the confidence of the predictions that can be made. Similarly, coordinated changes in expression of a greater number of the identified genes indicates increases the confidence with which predictions can be made. As more polymorphisms of the genes listed in Table 1 are identified, even more powerful risk profiling will be possible. Thus, in other embodiments of the invention the expression of at least five genes or at least about five genes is assayed to determine the capacity of collateral development. In yet further embodiments the number of genes assayed is ten. In yet other embodiments the number of genes assayed is 20 or at least about 20. In still yet other embodiments the number of genes assayed is 50 or at least about 50.

The skilled artisan will recognize that, due to the heterogeneous nature of collateral development, not all individuals with poor collateral development will exhibit altered expression of every last one of the genes listed in Table 1. Thus, it is possible that one, a few, or many genes will not exhibit significantly altered expression (and therefore will contain no biologically important polymorphisms or altered DNA methylation patterns), and that different individuals will exhibit different combinations; yet, the coordinated changes induced by the polymorphisms in the expression of the totality of genes are highly predictive of the presence of prediction of poor vs good collateral development.

In general, where the expression of only a relatively small number of genes is studied, changes in expression in most or all of the genes can be observed to provide a reliable diagnosis of good vs poor collateral development. For example, where only three genes are measured, all three genes can show relevant changes in expression to permit a reliable diagnosis impaired collateral development. Where five genes are studied, changes in at least four genes typically will provide a reliable diagnosis. Where ten genes are measured, a reliable diagnosis is obtained where changes in at least seven genes are observed. Where more than 10 genes are measured, changes in 90%, 80%, 70%, 60% or 50% of the measured genes are predictive of impaired collateral development. As these percentages decrease, the reliability of the diagnosis also decreases, but the skilled worker will recognize that when a coordinated change in expression of 20 or 30 genes of the genes listed in Table 1 is observed this is highly predictive of the likelihood of poor vs good collateral development. In general, as the number of genes increases, it is possible to provide a reliable diagnosis by observing coordinated changes in expression in a relatively smaller subset of the genes studied.

Tissues Sampled to Determine Altered Gene Expression and the Presence of Polymorphisms that Cause Biologicallv Important Alterations in Relevant Gene Expression

Although any sample containing nucleic acid would be appropriate for this purpose, the simplest tissue to sample is peripheral venous or arterial blood. However, other tissues may be used, such as vascular tissue, in particular arterial vascular tissue or venous vascular tissue.

Methods of Studying Gene Polymorphisms, DNA methylation patterns, and protein levels of the Genes Listed in Table 1

Polymorphisms can be identified by several methods including restriction enzyme digestion, sequencing, short tandem repeat association studies, single nucleotide polymorphism association studies, etc. These methods are well-known in the art.

Gene expression can also be studied at the protein level. Target tissue is first isolated and then total protein is extracted by well known methods. Quantitative analysis is achieved, for example, using ELISA methods employing a pair of antibodies specific to the target protein(s).

A subset of the proteins listed in Table 1 are soluble or secreted. In such instances the proteins may be found in the blood, plasma or lymph and an analysis of those proteins may be afforded by any of those methods described for the analysis of proteins in such tissues. This provides a minimally invasive means of obtaining patient samples for predicting the ability to generate collaterals. Methods for identifying secreted proteins are known in the art.

Gene polymorphisms are detected reliably with tissue derived from any source, including peripheral blood; blood protein levels can serve as a source of identifying altered gene expression.

RNA Expression

Methods of isolating RNA from tissue are well known in the art. See, for example, Sambrook et al. Molecular Cloning: A Laboratory Manual (Third Edition) Cold Spring Harbor Press, 2001. Commercial reagents also are available for isolating RNA.

Briefly, for example, cells or tissue are lysed and the lysed cells centrifuged to remove the nuclear pellet. The supematant is then recovered and the nucleic acid extracted using phenol/chloroform extraction followed by ethanol precipitation. This provides total RNA, which can be quantified by measurement of optical density at 260-280 nM.

mRNA can be isolated from total RNA by exploiting the “PolyA” tail of mRNA by use of several commercially available kits. QIAGEN mRNA Midi kit (Cat. No. 70042); Promega PolyATtract® mRNA Isolation Systems (Cat. No. Z5200). The QIAGEN kit provides a spin column using Oligotex Resin designed for the isolation of poly A mRNA and yields essentially pure mRNA from total RNA within 30 minutes. The Promega system uses a biotinylated oligo dT probe to hybridize to the mRNA poly A tail and requires about 45 minutes to isolate pure mRNA.

mRNA can also be isolated by using the cesium chloride cushion gradient method. Briefly the flash frozen tissue if homogenized in Guanethedium isothiocyanate, layered over a cushion of cesium chloride and ultracentrifuged for 24 hours to obtain the total RNA.

Genetic Microarray Analysis

Microarray technology is an extremely powerful method for assaying the expression of multiple genes in a single sample of mRNA. For example, Gene Chip® technology commercially available from Affymetrix Inc. (Santa Clara, Calif.) uses a chip that is that is plated with probes for over thousands of known genes and expressed sequence tags (ESTs). Biotinylated cRNA (linearly amplified RNA) is prepared and hybridized to the probes on the chip. Complementary sequences are then visualized and the intensity of the signal is commensurate with the number of copies of mRNA expressed by the gene.

Protein Expression

Gene expression may also be studied at the protein level. Target tissue is first isolated and then total protein is extracted by well known methods. Quantitative analysis is achieved, for example, using ELISA methods employing a pair of antibodies specific to the target protein.

A subset of the proteins listed in Table 1 are soluble or secreted. In such instances the proteins may be found in the blood, plasma or lymph and an analysis of those proteins may be afforded by any of those methods described for the analysis of proteins in such tissues. This provides a minimally invasive means of obtaining patient samples for estimate of risk of developing restenosis or of atherosclerosis. Methods for identifying secreted proteins are known in the art.

The emerging technology of proteomics can supply a powerful analytic tool to assay for changes in large numbers of proteins.

The following examples are offered to illustrate embodiments of the present invention, but should not be viewed as limiting the scope of the invention.

### EXAMPLES

### Microarray Analysis of the Mouse Hindlimb

Isolation of RNA

Mice underwent femoral artery ligation and extirpation.A control group was treated by sham surgery. Mouse adductor muscles after surgery and sham surgery were collected and flash frozen. Pooled muscles (30-50 mg) were crushed into powder using a mortar and pestle (collected with liquid nitrogen) and then homogenized in 2.5 ml of guanidinium isothiocyanate. Total RNA was extracted using ultracentrifugation on cesium chloride cushion gradient for 24 hours at 4° C. See Sambrook et al supra.

Target Preparation and DNA Microarray Hybridizations

For the first strand cDNA synthesis reaction, 5.0-8.0 μg of total RNA was incubated at 70° C. for 10 minutes with T7-(dT) 24 primer, then placed on ice. For the temperature adjustment step, 5X first stand cDNA buffer, 0.1 M DTT, and 10 mM dNTP mix was added and the reaction incubated for 1 hour at 42° C. SSII reverse transcriptase was added, and the reaction incubated for 1 hour at 42° C. With the first strand synthesis completed, 5X second strand reaction buffer, 10 mM dATP, dCTP, dGTP, dTTP, DNA Ligase, DNA Polymerase I, and RNaseH were added to the reaction tube. Samples were then incubated at 16°. Following the addition of 0.5 M EDTA, cDNA was cleaned using phase lock gels-phenol/chloroform extraction, followed by ethanol precipitation.

Synthesis of Biotin-Labeled CRNA (In vitro transcription)

The synthesis of biotin-labeled cRNA was completed using the ENZO BioArray RNA transcript labeling kit from (ENZO Biochem, Inc., New York, N.Y.) according to the manufacturers protocol. To set up the reaction 1 μg of cDNA, 10X HY reaction buffer, 10X Biotin labeled ribonucleotides, 10X DTT, 10X RNase inhibitor mix and 20X T7 RNA polymerase were incubated at 37° C. for 4-5 hours. RNeasy spin columns from QIAGEN were used to purify the labeled RNA, followed by ethanol precipitation and quantification.

Fragmentation of cRNA for Target Preparation

5X fragmentation buffer (200 mM Tris-acetate, pH 8.1, 500 mM KOAc, 150 mM Mg)Ac) was added to the cRNA. Samples were incubated at 94° C. for 35 minutes, then placed on ice. Fragmented cRNA was stored at −70° C.

Target Hybridization

Hybridization cocktail was prepared as follows: fragmented cRNA (15 μg adjusted), control oligonucleotide B2 (Affymetrix), 20X eukaryotic hybridization controls (Affymetrix), herring sperm DNA, acetylated BSA, and 2X hybridization buffer (Affymetrix) were combined, and heated to 99° C. for five minutes. Hybridization cocktail was then centrifuged at maximum speed for five minutes to remove any insoluble materials from the mixture. Following centrifugation, cocktail was heated at 45° C. for five minutes. The clarified hybridization cocktail was then added to the Affymetrix probe array cartridge that had been pre-wet with 1X hybridization buffer. The probe array was then placed in a 45° C. rotisserie box oven set at 60 rpm and hybridized for 16 hours.

Washing, Staining and Scanning Probe Arrays

The GeneChip® Fluidics Station 400 was used to wash and stain the array. This instrument was run using GeneChip® software. Briefly, arrays were washed for 10 cycles with non-stringent wash buffer at 25° C., followed by 4 cycles of washing with stringent wash buffer at 50° C. The array was then stained for 10 minutes with Phycoerythrin-streptavidin at 25° C. The array was then washed for 10 cycles with non-stringent wash buffer at 25° C. The probe array was the stained again with phycoerythrin-streptavidin for 10 minutes at 25° C., and then washed for 15 cycles with non-stringent wash buffer at 30° C. Hybridization signals are detected by placing the probe array in an HP Gene Array™ Scanner, which operated using GeneChip® software.

Data Analysis

Data analysis was performed using GeneChip® software (version 3.3) using the manufacturer's instructions. Lockhart, D. J. et al., Nat. Biotechnol. 14:1675-80 (1996). Briefly, each gene was represented and queried by 1-3 probe sets on the chip. Each probe set comprises 16 perfect match (PM) and 16 mismatch (MM) 25 nucleotide base probes. The mismatch has a single base change in the middle of the 25 base pair probe. The hybridization signal from the PM and the MM probes were compared and this allowed for a measure of signal intensity that is specific and eliminated the nonspecific cross hybridization from the data of the two control chips. Intensity differences as well as ratios of intensity of each probe pair are used to make a “present” or “absent” call. The controls were used as baseline and the experimental GeneChip® assay values compared to the base line to derive four matrixes which were used to determine the difference calls that indicate whether the transcription level of a particular gene is changed.

Iterative comparisons were performed using a spreadsheet analysis (Microsoft Excel). Each experimental data set at a particular time point (n=2) and the difference in expression between the controls and experimental was determined for each gene. Genes with a consistent difference call across all four pairwise comparisons were extracted for further analysis.

GeneSpring® Analysis

The data from each GeneChip® assay was fed into the GeneSpring® software and clustering of genes based on their temporal expression profile was analyzed. Correlation coefficients of 0.97 or greater were taken as a cutoff to create gene-clusters with significant expression homology.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A method for predicting the likelihood that a subject will develop collaterals, comprising assaying the expression level of at least three in genes in said subject. in a sample obtained from said mammal.

2. The method according to claim 1, wherein the likelihood of collateral development is predicted by the altered expression of at least three, at least five, at least ten, at least twenty genes, or at least twenty genes in said sample.

3. The method according to claim 1, wherein the likelihood of collateral development is predicted by increased expression of at least three, at least five, at least ten, at least twenty genes, or at least twenty genes in said sample.

4. The method according to claim 1, wherein the likelihood of collateral development is predicted by decreased expression of at least three, at least five, at least ten, at least twenty genes, or at least twenty genes in said sample.

5. The method according to claim 1 or claim 2, wherein said genes are selected from the genes listed in Table 1.

6. The method according to claim 3, wherein said genes are selected from the genes listed in Table 2.

7. The method according to claim 4, wherein said genes are selected from the genes listed in Table 2.

8. The method according to claim 1 wherein said sample comprises blood from said subject.

9. The method according to claim 1, wherein said altered expression level is at least two fold higher or lower than a reference level.

10. The method of any of claims 1-9 wherein the level of gene expression is determined by assaying the level of protein expression in a sample.

11. A method for predicting the likelihood that a subject will develop collaterals, comprising detecting the presence of at least three genetic variations in a sample from said patient, wherein said genetic variations are SNPs or altered DNA methylation patterns.

12. The method according to claim 11, wherein the likelihood of collateral development is predicted by the presence of genetic variations in at least three, at least five, at least ten, at least twenty genes, or at least twenty genes in said sample.

13. The method according to claim 11 or 12, wherein said genes are selected from the group consisting of the genes listed in Table 1.

14. The method according to claim 1 or claim 11 wherein the method of assay comprises using a genetic microarray or quantitative PCR.

15. The method according to claim 11 wherein the assay comprises a method to detect DNA methylation patterns.

16. The method according to claim 11 wherein the assay comprises a method to detect single nucleotide polymorphisms.

17. A kit for carrying out the assay according to claim 1 or claim 11, wherein said assay is to be carried out using a PCR and wherein said kit comprises a set of primers suitable for amplifying at least three, at least five, at least ten, or at least twenty DNA or RNA sequences corresponding to the genes in Table 1.

18. A kit for carrying out the assay according to claim 11 wherein said kit comprises a nucleic acid array capable of detecting single nucleotide polymorphisms in a plurality of the genes identified in Table 1.

19. A kit according to claim 18 wherein said array is capable of detecting single nucleotide polymorphisms, if present, in a majority of the genes identified in Table 1.

20. The method according to claim 1, wherein the expression level of said genes is determined by measuring the concentration of the proteins encoded by said genes.

21. The method according to claim 20, wherein said proteins are soluble proteins.

22. The method according to claim 21, wherein said sample is blood and/or lymph.

23. The method according to claim 20, wherein the level of protein expressions is determined by ELISA.

24. A method of promoting collateral formation in a subject, comprising administering to said subject a composition that decreases expression of at least one gene identified in Table 2 and/or that increases expression of at least one gene identified in Table 3.

25. The method according to claim 24, wherein said composition comprises an antisense oligonucleotide, an siRNA molecule, an RNAi molecule, an oligonucleotide that binds to mRNA to form a triplex, or a DNA molecule that is transcribed in said subject to produce an antisense oligonucleotide, an siRNA molecule, an RNAi, or an oligonucleotide that binds to mRNA to form a triplex.

26. The method according to claim 24, wherein said composition comprises an antibody or a soluble protein receptor that binds to a protein that inhibits collateral formation in said subject.

27. The method according to claim 26, wherein said composition comprises a human antibody or a human soluble protein receptor.

28. The method according to claim 24, wherein said composition comprises a protein that is administered to supplement the loss of a protein encoded by a gene identified in Table 3.
